(19) United States
(12) Patent Application Publication
(10) Pub. No.: US 2011/0207741 A1
(43) Pub. Date: Aug. 25, 2011

(54) Activation of the Renin-Angiotensin System (RAS) and Sudden Cardiac Death

(75) Inventor: Samuel Dudley, Chicago, IL (US)

(73) Assignee: THE BOARD OF TRUSTEES OF THE UNIVERSITY OF ILLINOIS, Urbana, IL (US)

(21) Appl. No.: 13/032,629

(22) Filed: Feb. 22, 2011

Related U.S. Application Data
(60) Provisional application No. 61/306,050, filed on Feb. 19, 2010.
(60) Provisional application No. 61/373,002, filed on Aug. 12, 2010.

(51) Int. Cl. A61K 31/517; A61K 31/506; A61K 31/496; A61K 31/519; A61K 31/52; A61K 31/4375; A61K 31/47; A61K 31/495; A61K 31/4164; A61P 9/00; A61P 9/10; A61P 9/04; A61P 3/00; A61P 13/12; A61P 9/12; A61P 3/10; A61P 3/04

(52) U.S. Cl. 514/252.17; 514/252.19; 514/252.18; 514/275; 514/253.06; 514/265.1; 514/262.1; 514/263.4; 514/300; 514/264.1; 514/264.11; 514/311; 514/255.01; 514/399; 514/266.22

(57) ABSTRACT

Provided herein are methods of treating a medical condition in which RAS activation is increased. The method comprises the step of administering to a subject a c-Src inhibitor in an amount effective to treat the medical condition. The invention also provides a method of treating or preventing a cardiac arrhythmia. The method comprises the step of administering to the subject a c-Src inhibitor in an amount effective to treat or prevent the cardiac arrhythmia. The invention additionally provides a method of delaying the onset of sudden cardiac death. The method comprises the step of administering to the subject a c-Src inhibitor in an amount effective to delay the onset of SCD. Methods of augmenting gap junction function and methods of increasing Connexin 43 levels in a subject in need thereof are further provided.

### CROSS-REFERENCE TO RELATED APPLICATIONS

This application claims priority to U.S. Provisional Patent Application No. 61/306,050, filed on Feb. 19, 2010, and U.S. Provisional Patent Application No. 61/373,002, filed on Aug. 12, 2010, each of which is incorporated by reference in their entirety.

### GRANT FUNDING

This invention was made with government support under Grant Nos. R01 HL085558; R01 HL073753 R01 HL058000, awarded by the National Institutes of Health. The government has certain rights in the invention.

### BACKGROUND

Sudden cardiac death (SCD) accounts for approximately 325,000 deaths per year in the United States, which number is higher than the number of deaths attributed to lung cancer, breast cancer, or acquired immune deficiency syndrome (AIDS). SCD is responsible for about 50% of deaths from heart failure and often is the first expression of coronary disease. See, Sovari et al., “Sudden Cardiac Death,” e-medicine Cardiology, article 151907, updated Nov. 4, 2010; and Zheng et al., Circulation 104: 2158-2163 (2001). A common cause of SCD is ventricular arrhythmia, including, for example, ventricular tachycardia (VT), in which the resting heart rate is faster than normal, ventricular fibrillation (VF), in which there is uncoordinated contraction of the cardiac muscle of the ventricles in the heart, making the muscles quiver rather than contract properly, or an arrhythmic condition in which both VT and VF are present. See, Wedro, B., “Sudden Cardiac Arrest (Sudden Cardiac Death),” medicine.net, Kulick and Soppler, eds. Current methods of treating ventricular fibrillation include defibrillation via an electrical defibrillator or a precordial thump. Anti-arrhythmic drugs aim to treat or prevent ventricular arrhythmias, thereby, preventing SCD. However, it has been reported that some anti-arrhythmic agents can actually increase the risk of SCD.

### SUMMARY

Presented herein for the first time are data demonstrating that administration of a c-Src inhibitor to a subject exhibiting increased renin-angiotensin system (RAS) activation (e.g., a subject exhibiting increased levels of ACE or angiotensin II) prevents SCD and ventricular arrhythmias in the subject.

Accordingly, the invention provides a method of treating a subject exhibiting increased levels of RAS activation (e.g., a subject exhibiting increased levels of ACE or angiotensin II). The method comprises the step of administering to the subject a c-Src inhibitor. In exemplary aspects, the subject is administered a c-Src inhibitor in an amount effective to treat a medical condition in which RAS activation is increased (e.g., a medical condition in which ACE or angiotensin II is increased). Accordingly, the invention also provides a method of treating a medical condition in which RAS activation is increased (e.g., a medical condition in which ACE or angiotensin II levels are increased). The method comprises the step of administering to the subject a c-Src inhibitor in an amount effective to treat the medical condition. In exemplary aspects, the medical condition in which RAS activation is increased is a cardiac condition, a metabolic disease, or a renal disease, as further described herein.

The invention also provides a method of treating or preventing a cardiac arrhythmia in a subject in need thereof. The method comprises the step of administering to the subject a c-Src inhibitor in an amount effective to treat or prevent a cardiac arrhythmia. In exemplary aspects, the cardiac arrhythmia is a ventricular arrhythmia. In exemplary aspects, the cardiac arrhythmia is an atrial arrhythmia.

The invention further provides a method of delaying the onset of SCD in a subject in need thereof. The method comprises the step of administering to the subject a c-Src inhibitor in an amount effective to delay the onset of SCD.

Also presented herein for the first time are data which demonstrates that administration of a c-Src inhibitor to a subject exhibiting increased RAS activation improves gap junction function in the cardiac muscle of the subject. Accordingly, the invention provides a method of augmenting gap junction function in cardiac muscle of a subject in need thereof. The method comprises the step of administering to the subject a c-Src inhibitor in an amount effective to augment gap junction function in cardiac muscle of the subject. In exemplary aspects, the subject exhibits increased levels of RAS activation (e.g., a subject exhibiting increased levels of ACE or angiotensin II). In exemplary aspects, the subject is at risk for sudden cardiac death. In exemplary aspects, the subject is suffering from a cardiac arrhythmia or is at risk for a cardiac arrhythmia.

Further presented herein for the first time are data which demonstrate that administration of a c-Src inhibitor to a subject exhibiting increased RAS activation increases levels of the gap junction protein Connexin 43. Accordingly, the invention additionally provides a method of increasing Connexin 43 levels in a subject in need thereof. The method comprises the step of administering to the subject a c-Src inhibitor in an amount effective to increase Connexin 43 levels. In exemplary aspects, the Connexin 43 levels are cardiac Connexin 43 levels, e.g., ventricular Connexin 43 levels, Connexin 43 levels in the gap junction.

Because a reduction or lack of Connexin 43 can result in slow conduction velocity and ventricular arrhythmia, the method of increasing Connexin 43 levels provided herein may achieve an increase in conduction velocity and treat or prevent a ventricular arrhythmia. Therefore, the invention further provides a method of increasing conduction velocity in a subject in need thereof. The method comprises the step of administering to the subject a c-Src inhibitor in an amount effective to increase the conduction velocity in the subject. The invention furthermore provides a method of treating or preventing a ventricular arrhythmia in a subject in need thereof. The method comprises the step of administering to the subject a c-Src inhibitor in an amount effective to treat or prevent ventricular arrhythmia in the subject.

### BRIEF DESCRIPTION OF THE DRAWINGS

FIG. 1 represents a survival analysis and inducibility of ventricular tachycardia (VT). (A) Kaplan-Meier survival distribution analysis of ACE8/8 untreated mice, ACE8/8 mice that received PP3 treatment, ACE8/8 mice that received PP1 treatment, and wild-type mice that received PP1 treatment. PP1 treatment significantly reduced the sudden cardiac death rate with the mean survival time of 10.2+/−1.5 days in ACE8/8 and 24.7+/−0.2 days in ACE8/8 treated with PP1 (P<0.0001, log-rank test). No death happened in WT mice receiving PP1 treatment. PP3, the ineffective analog of PP1, did not increase the survival rate in ACE8/8 mice (11.2+/−1.2 days vs. 10.2+/−1.5 days, p=0.085). (B) Inducibility of VT was tested using a 1.1 F catheter and an internal jugular vein access. A rhythm with more than 3 consecutive ventricular beats was considered to be VT. Examples of intracardiac electrograms of WT, ACE8/8 and ACE8/8 treated with PP1 are shown after 12 beats of burst pacing with pacing cycle length of 50 ms. VT inducibility rate was statistically reduced with PP1 treatment in ACE8/8 mice (86.9% vs. 50%, P=0.03).

FIG. 2 represents a Western blot analysis of protein levels. All results are corrected for the GAPDH level. (A) The total c-Src protein level was 1.47-fold higher in ACE8/8 mice than in wild-type mice (P=0.007), and the phospho-(Tyr416) c-Src protein level was 2.6-fold higher in ACE8/8 mice than in control mice (P=0.01). The Cx43 level was reduced in ACE8/8 mouse hearts to 36% of the level in wild-type mouse hearts (P=0.0001). (B) PP1 treatment: Four weeks of intraperitoneal injection of PP1 in ACE8/8 mice decreased the total c-Src protein level to 58% of that in untreated ACE8/8 mice (P=0.03) and reduced the phospho-(Tyr 416) c-Src level to 75% of that in untreated ACE8/8 mice (P=0.04). More importantly, PP1 treatment resulted in a 2.1-fold increase in the Cx43 level in ACE8/8 mice and increased the level of Cx43 to 68% of its normal level in wild-type mice.

FIG. 3 represents results of immunostaining for Cx43. The Cx43 level which was measured as the ratio of stained area for Cx43 in the field of view to the total field of view, was significantly lower in ACE8/8 mouse hearts than in wild-type mouse hearts (18.3±0.6% vs. 5.4±0.7%, P=0.0001). PP1 treatment resulted in a 2-fold increase in the Cx43 level in ACE8/8 mouse hearts (P=0.0005). The result of immunostaining for Cx43 was consistent with the result of western blotting and it confirmed that the improvement in the Cx43 protein level was associated with the increased level of this protein at its site of function, gap junctions. Treatment with PP3, the inactive analog of PP1, did not increase Cx43 level in compare to untreated ACE8/8 mice (5.4+/−0.7% vs. 6.5+/−1%, P=0.19).

FIG. 4 represents an assessment of the function of gap junctions via modified scrape loading. Lucifer Yellow (LY; 400 kDa) spreads extensively through myocytes from WT hearts (A), far beyond the spread of TRD (B; 10000 kDa). Overlay image is seen in C. In the ACE8/8 hearts LY spread is decreased (D) with fewer cells coupled beyond the TRD (E). Overlay image indicates that fewer cells are coupled in these ACE8/8 hearts (F). Treatment of mice with PP1 restores normal dye spread (G) to well beyond the TRD (H). Note that the overlay image from WT mice (C) and from the PP1 treated mice (I) are similar in the extent of dye spread seen. Quantification of the overall dye spread shows that the overall dye spread in the ACE8/8 mice is significantly decreased (0.14±0.01 mm vs. 0.21±0.02 mm respectively, P=0.01) and that is restored to the normal level after PP1 treatment (0.21±0.02 mm in WT vs. 0.20±0.02 mm in PP1, P=0.84, PP1 vs. ACES/8 P=0.01). Analysis of the anisotropy of dye spread indicates that in this model dye spread is lost in both the transverse and longitudinal directions, although more prominent in the longitudinal direction (K).

FIG. 5 represents a schematic figure of the relation between Ang II, c-Src and Cx43. c-Src mediates the effect of Ang II on Cx43 reduction and impaired gap junction function. PP1 is a known specific inhibitor of c-Src tyrosine kinase that interrupts Ang II mediated Cx43 reduction, myocyte uncoupling and sudden arrhythmic death.

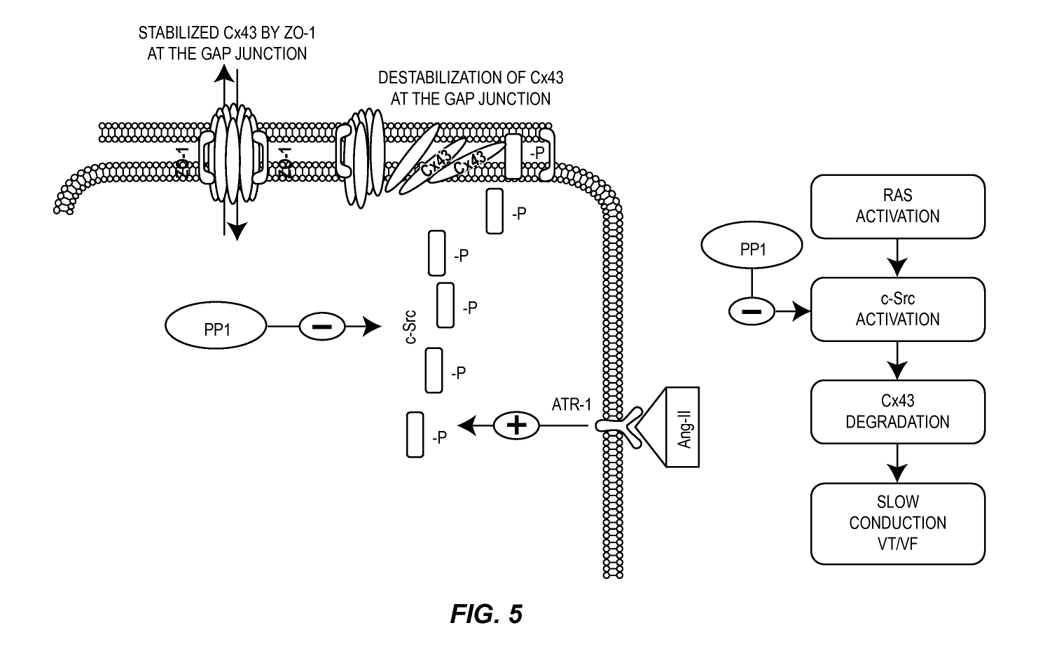

### DETAILED DESCRIPTION

RAS Activation and Related Medical Conditions

The invention provides a method of treating a subject exhibiting increased levels of RAS activation. The method comprises the step of administering to the subject a c-Src inhibitor. As used herein, the term “renin-angiotensin system” or “RAS” is synonymous with the term “renin-angiotensin-aldosterone system” or “RAAS” and refers to a set of biological pathways that are activated in response to decreased blood volume. When blood volume is low, the kidneys produce renin, which stimulates the production of angiotensin from angiotensinogen. Angiotensin in turn causes vasoconstriction, resulting in increased blood pressure. Angiotensin also causes secretion of aldosterone from the adrenal cortex. Aldosterone causes the tubules of the kidneys to increase reabsorption of sodium and water into the blood, which increases the volume of fluid in the body, thereby increasing blood pressure. Angiotensin II is created upon the removal of two C-terminal residues of angiotensin I by angiotension coverting enzyme (ACE).

In exemplary embodiments, a subject exhibiting increased levels of RAS activation is a subject exhibiting an increased level of a positive RAS marker, a decreased level of a negative RAS marker, or a combination thereof. As used herein, the term “positive RAS marker” refers to a marker of which the level (e.g., expression level, protein level, mRNA level) or activity (e.g., enzyme) increases in response to RAS activation. In exemplary aspects, the positive RAS marker is renin, angiotensin II, aldosterone, angiotensin converting enzyme (ACE), NADPH oxidase, or a combination thereof. In exemplary aspects, the positive RAS marker is ACE (e.g., ACE protein, ACE activity, ACE mRNA) or angiotensin II (e.g., angiotensin II protein, angiotensin II mRNA, angiotensin II activity). Accordingly, in exemplary aspects, subject exhibiting increased levels of RAS activation is a subject exhibiting increased levels of ACE (e.g., ACE protein, ACE activity, ACE mRNA) or angiotensin II (e.g., angiotensin II protein, angiotensin II mRNA, angiotensin II activity).

As used herein, the term “negative RAS marker” refers to a marker of which the level or activity decreases in response to RAS activation. In exemplary aspects, the negative RAS marker is nitric oxide, angiotensin I, or angiotensinogen.

In some embodiments, the increased levels of RAS activation is at least or about a 1.5 fold increase in RAS activation, as compared to a control subject. The increased levels of RAS activation in exemplary aspects is at least or about a 2-fold increase, at least or about a 3-fold increase, at least or about a 5-fold, at least or about a 10-fold increase, at least or about a 25-fold increase, at least or about a 50-fold increase, at least or about a 75-fold increase, at least or about a 100-fold increase, or more. The control subject in some aspects is a subject known to not exhibit increased RAS activation levels, e.g., a subject with a normal body mass index (BMI), a subject not suffering from any of the medical conditions in which RAS activation is known to be elevated (e.g., diabetes, hypertension, obesity, MI, heart failure). In some embodiments, the increase in RAS activation is a sustained increase in RAS activation, wherein the increase is exhibited by the subject for a time period of at least 12 hours, 24 hours, 2 days, 5 days, 7 days, 2 weeks, 4 weeks, 2 months, 4 months, 6 months, 10 months, 1 year, 2 years, 3 years, or more. Methods of measuring an increase in RAS activation in cells of a subject are known in the art and include, but not limited to, assaying for increases of positive RAS markers or decreases in negative RAS markers via immunoassays (e.g., Western blotting, immunohistochemistry, immunofluorescence, radio immunoassays (RIA), and the like) and quantitative PCR. Suitable methods include those set forth in Example 1.

In exemplary aspects, the subject exhibiting increased levels of RAS activation is a subject comprising cardiac cells exhibiting increased levels of RAS activation. In exemplary aspects, RAS activation is increased only in the cardiac cells of the subject. In exemplary aspects, the subject exhibiting increased levels of RAS activation is a subject comprising cardiac cells exhibiting increased levels of ACE or angiotensin II. In exemplary aspects, increased levels of ACE or angiotensin II are exhibited by only cardiac cells of the subject.

In exemplary aspects, the subject is a subject at risk for SCD. In exemplary aspects, the subject is any of the subjects described in the section entitled “Sudden cardiac death.” In exemplary aspects, the subject is a subject with a structural abnormality of the heart, a congenital heart defect, or a metabolic disease.

In other exemplary aspects, the subject is a subject that does not suffer from a structure heart abnormality. In exemplary aspects, the subject has a normal left ventricular ejection fraction (e.g., a left ventricular ejection fraction of about 45% or more), does not suffer from ventricular fibrosis, does not suffer from hypertension, or a combination thereof. In exemplary aspects, the subject exhibits cardiac oxidative stress, abnormal gap junction function, impaired (e.g., slowed) cardiac conduction (e.g., slow conduction velocity), reduced Connexin 43 levels (e.g., reduced ventricular Connexin 43 levels), increased RAS activation, increased angiotensin II levels, increased angiotensin converting enzyme (ACE) (e.g., increased ACE levels in cardiac cells), increased c-Src levels (e.g., increased total c-Src levels, increased phospho-c-Src levels), reduced myocyte coupling, or a combination of the foregoing.

In exemplary embodiments, the subject suffers from a cardiac arrhythmia at the time of administering the c-Src inhibitor. The cardiac arrhythmia in some aspects is an arrhythmia described herein, e.g., a ventricular arrhythmia (e.g., a ventricular tachycardia (VT), ventricular fibrillation (VF), or an arrhythmic condition in which both VT and VF are present), an atrial arrhythmia. In exemplary embodiments, the subject does not suffer from a cardiac arrhythmia at the time of administering the c-Src inhibitor.

In exemplary embodiments, the subject has a medical history of cardiac arrhythmia, e.g., a ventricular arrhythmia (e.g., a ventricular tachycardia (VT), ventricular fibrillation (VF), or an arrhythmic condition in which both VT and VF are present). In exemplary embodiments, the subject does not have a medical history of a cardiac arrhythmia, e.g., a ventricular arrhythmia (e.g., a ventricular tachycardia (VT), ventricular fibrillation (VF), or an arrhythmic condition in which both VT and VF are present). In exemplary embodiments, the subject is at risk for a cardiac arrhythmia, as described herein. In exemplary aspects, the subject is any of the subject described in the section entitled “Cardiac Arrhythmias.”

In exemplary aspects, the subject is administered a c-Src inhibitor in an amount effective to treat a medical condition in which RAS activation is increased (e.g., a medical condition in which ACE or angiotensin II is increased). Accordingly, the invention provides a method of treating a medical condition in which RAS activation is increased (e.g., a medical condition in which ACE or angiotensin II levels are increased). The method comprises the step of administering to the subject a c-Src inhibitor in an amount effective to treat the medical condition.

In exemplary embodiments, the medical condition is a cardiac condition. In exemplary aspects, the cardiac condition is heart failure. Heart failure (HF) is defined as the ability of the heart to supply sufficient blood flow to meet the body's needs. In some embodiments, the signs and symptoms of heart failure include dyspnea (e.g., orthopnea, paroxysmal nocturnal dyspnea), coughing, cardiac asthma, wheezing, dizziness, confusion, cool extremities at rest, chronic venous congestion, ankle swelling, peripheral edema or anasarca, nocturia, ascites, heptomegaly, jaundice, coagulopathy, fatigue, exercise intolerance, jugular venous distension, pulmonary rales, peripheral edema, pulmonary vascular redistribution, interstitial edema, pleural effusions, or a combination thereof. In some embodiments, the symptom of heart failure is one of the symptoms listed in the following table, which provides a basis for classification of heart failure according to the New York Heart Association (NYHA).

| NYHA | |
|---|---|
| Class | Symptoms |
| I | No symptoms and no limitation in ordinary physical activity, |
| | e.g. shortness of breath when walking, climbing stairs etc. |
| II | Mild symptoms (mild shortness of breath and/or angina) and |
| | slight limitation during ordinary activity. |
| III | Marked limitation in activity due to symptoms, even during |
| | less-than-ordinary activity, e.g. walking short distances |
| | (20-100 m). |
| | Comfortable only at rest. |
| IV | Severe limitations. Experiences symptoms even while at rest. |
| | Mostly bedbound patients. |

In exemplary aspects, the cardiac condition treated by the method of the invention is a systolic heart failure, which is heart failure caused or characterized by a systolic dysfunction. In simple terms, systolic dysfunction is a condition in which the pump function or contraction of the heart (i.e., systole), fails. Systolic dysfunction may be characterized by a decreased or reduced ejection fraction, e.g., an ejection fraction which is less than 45%, and an increased ventricular end-diastolic pressure and volume. In some aspects, the strength of ventricular contraction is weakened and insufficient for creating an appropriate stroke volume, resulting in less cardiac output. In some aspects, the systolic heart failure is an ischemic heart failure. In alternative aspects, the systolic heart failure is a nonischemic heart failure.

In other exemplary aspects, the cardiac condition treated by the method of the invention is a diastolic heart failure in which there is the ventricle fails to adequately relax and typically denotes a stiffer ventricular wall. Diastolic heart failure is described further in Zile et al., JACC 41: 1519-1522 (2003).

In exemplary aspects, the cardiac condition treated by the method of the invention is a condition precursory to heart failure. In exemplary aspects, the medical condition is a low ejection fraction, e.g., less than 55%, less than 50%, less than 45%.

In exemplary embodiments, the cardiac condition treated by the method of the invention is a cardiac arrhythmia, such as a ventricular arrhythmia or an atrial arrhythmia. The cardiac arrhythmia may be any of those described herein, e.g., ventricular tachycardia, ventricular fibrillation, atrial fibrillation, atrial tachycardia.

In exemplary embodiments, the cardiac condition treated by the method of the invention is a condition precursory to a cardiac arrhythmia. In exemplary aspects, the medical condition is impaired cardiac electrical conduction or slowed conduction velocity in cardiac cells.

In exemplary embodiments, the cardiac condition treated by the method of the invention is myocardial infarction (MI). In exemplary aspects, the MI is an acute transmural MI or an acute subendocardial MI. In some aspects, the MI is a Type 1 MI, Type 2 MI, Type 3 MI, Type 4 MI (e.g., Type 4a MI, Type 4b MI), or Type 5 MI, as classified in Thygesen et al., Eur Heart 28(20): 2525-2528 (2007).

In exemplary embodiments, the cardiac condition treated by the method of the invention is atherosclerosis, also known as arteriosclerotic vascular disease or ASVD.

In exemplary embodiments, the cardiac condition treated by the method of the invention is a cardiomyopathy. In some aspects, the cardiomyopathy is an intrinsic cardiomyopathy (e.g., hypertrophic cardiomyopathy, arrhythmic right ventricular cardiomyopathy, isolated ventricular non-compaction, mitochondrial myopathy, dilated cardiomyopathy, restrictive cardiomyopathy, Takotsubo cardiomyopathy, Loeffler endocarditis). The cardiomyopathy in some aspects is an extrinsic cardiomyopathy (e.g., diabetic cardiomyopathy, age-related cardiomyopathy, alcoholic cardiomyopathy, ischemic cardiomyopathy, a cardiomyopathy caused by amyloidosis, hemochromatosis, Chagas disease, hyperthyroidism, chemotherapy, muscular dystrophy, a nutritional disease).

In exemplary embodiments, the medical condition treated by the method of the invention is a congenital heart defect. In exemplary aspects, the congenital heart defect is a hypoplasia, an obstruction defect, a septal defect, or a cyanotic defect. In exemplary aspects, the congenital heart defect is selected from the group consisting of: Aortic stenosis, Atrial septal defect (ASD), Atrioventricular septal defect (AVSD), Bicuspid aortic valve, Dextrocardia, Double inlet left ventricle (DILV), Double outlet right ventricle (DORV), Ebstein's anomaly, Hypoplastic left heart syndrome (HLHS), Hypoplastic right heart syndrome (HRHS), Mitral stenosis, Pulmonary atresia, Pulmonary stenosis, Transposition of the great vessels, dextro Transposition of the great arteries (d-TGA), levo-Transposition of the great arteries (1-TGA), Tricuspid atresia, Persistent truncus arteriosus, Ventricular septal defect (VSD), Coarctation of the aorta (CoA), Interrupted aortic arch (IAA), Patent ductus arteriosus (PDA), Scimitar syndrome (SS), Partial anomalous pulmonary venous connection (PAPVC), Total anomalous pulmonary venous connection (TAPVC), tetralogy of Fallot (ToF), pentalogy of Cantrell, Shone's syndrome (also known as Shone's complex, Shone's anomaly).

In exemplary embodiments, the medical condition treated by the method of the invention is a metabolic disease, such as diabetes (e.g., Type 2 diabetes mellitus), obesity, hypertension, diabetic nephropathy, metabolic syndrome (also known as Syndrome X), insulin resistance, dyslipidemia, hyperglycemia, hyperinsulinemia, and the like. In exemplary aspects, the metabolic disease is a disease which is treatable by an angiotensin receptor blocker. See, for example, Prasad and Quyyumi, Circulation 110: 1507-1512 (2007).

In exemplary embodiments, the medical condition treated by the method of the invention is a renal disease. In exemplary aspects, the renal disease is acute renal failure, chronic kidney disease, renal interstitial fibrosis, diabetic nephopathy, glomerulonephritis, hydronephrosis, interstitial nephritis, kidney stones (nephrolithiasis), kidney tumors (e.g., Wilms tumor, renal cell carcinoma), lupus nephritis, minimal change disease, nephrotic syndrome, pyelonephritis, renal failure (e.g., other than acute renal failure and chronic kidney disease).

In exemplary embodiments, the medical condition treated by the method of the invention is characterized by one or more of: oxidative stress, inflammation, apoptosis, myocardial and renal fibrosis, hypertrophy, abnormal intracellular Ca²⁺ handling, abnormal potassium current, vasoconstriction, atherosclerosis, connexin reduction, sympathetic overactivity (through AT1R in central nervous system), insulin resistance, prothrombotic effect, and aging.

In exemplary embodiments, the medical condition treated by the method of the invention is a medical condition which is known to be treated by an angiotensin II blocking agent, including, but not limited to: diabetes mellitus, hypertension, systolic heart failure (e.g., ischemic heart failure, nonischemic heart failure), myocardial infarction, renal disease, stroke, arrhythmia in the setting of heart failure, dementia.

In exemplary embodiments, the medical condition is characterized by an increased risk of SCD and/or a cardiac arrhythmia. Accordingly, the invention provides a method of treating a medical condition characterized by an increased risk of SCD and/or a cardiac arrhythmia. The method comprises the step of administering to the subject a c-Src inhibitor in an amount effective to treat the medical condition. In exemplary aspects, the cardiac arrhythmia is a ventricular arrhythmia, e.g., a ventricular fibrillation, a ventricular tachycardia, an arrhythmic condition in which both ventricular fibrillation and ventricular tachycardia are present. In exemplary aspects, the medical condition characterized by an increased risk of SCD and/or a cardiac arrhythmia is selected from the group consisting of: cardiomyopathy, ischemic heart disease, congenital heart disease, heart failure (e.g., systolic heart failure, diastolic heart failure), inherited arrhythmic conditions.

With regard to the method of treating a medical condition in which RAS activation is increased provided herein, the subject treated may be any subject in need thereof. In exemplary aspects, the subject suffers from one of the medical conditions described herein at the time of administering the c-Src inhibitor. In exemplary aspects, the subject has a medical history of one of the medical conditions described herein. In exemplary aspects, the subject is at risk for one of the medical conditions described herein, and/or exhibits signs and symptoms of one of the medical conditions described herein.

Sudden Cardiac Death

As used herein, the term “sudden cardiac death” or “SCD” is synonymous with cardiac arrest, sudden arrest, sudden death, sudden cardiac arrest and refers to a medical condition characterized by a sudden loss of cardiac function. In SCD, the heart stops beating and pumping blood. SCD is further described in Zheng et al., Circulation 104: 2158-2163 (2001). In exemplary embodiments, the SCD is unexpected, occurring in a subject with an undiagnosed cardiac disease. In exemplary aspects, the SCD is sudden, occurring in a short period of time from the time of symptom onset, e.g., within 1 hour of symptom onset, within 45 minutes of symptom onset, within 30 minutes of symptom onset, with 15 minutes of symptom onset, within 10 minutes of symptom onset, within 5 minutes of symptom onset. If left untreated, the SCD in exemplary aspects will lead to a loss of consciousness, pulse, normal breathing and/or blood pressure, and eventually will lead to death.

The invention provides a method of preventing SCD in a subject in need thereof, e.g., a subject at risk for SCD. The method comprises the step of administering to the subject a c-Src inhibitor in an amount effective to prevent SCD in the subject.

As used herein, the term “prevent” and words stemming therefrom encompasses delaying the onset of the medical condition being prevented. Accordingly, the invention also provides a method of delaying the onset of SCD in a subject in need thereof, e.g., a subject at risk for SCD. The method comprises the step of administering to the subject a c-Src inhibitor in an amount effective to delay the onset of SCD in a subject. In exemplary aspects, the method delays the onset of SCD by 1 day, 2 days, 4 days, 6 days, 8 days, 10 days, 15 days, 30 days, two months, 4 months, 6 months, 1 year, 2 years, 4 years, or more. In exemplary aspects, delaying the onset of SCD is a delay in the first time occurrence of SCD.

As used herein, the term “prevent” and words stemming therefrom encompasses reducing the risk of the medical condition being prevented. The invention accordingly provides a method of reducing the risk of SCD in a subject in need thereof. The method comprises the step of administering to the subject a c-Src inhibitor in an amount effective to reduce the risk of SCD in the subject. In exemplary aspects, the method reduces the risk of SCD 2-fold, 5-fold, 10-fold, 20-fold, 50-fold, 100-fold, or more.

The invention further provides a method of increasing survival of a subject at risk for SCD. The method comprises the step of administering to the subject a c-Src inhibitor in an amount effective to increase survival of the subject. In exemplary aspects, the method increases survival of a subject at risk for SCD by 1 day, 2 days, 4 days, 6 days, 8 days, 10 days, 15 days, 30 days, two months, 4 months, 6 months, 1 year, 2 years, 4 years, or more.

The invention furthermore provides a method of treating a sign or symptom of sudden cardiac death in a subject in need thereof. The method comprises the step of administering to the subject a c-Src inhibitor in an amount effective to treat the sign or symptom. Signs and symptoms of SCD include, for example, a cardiac arrhythmia, e.g., a ventricular arrhythmia (e.g., ventricular fibrillation, ventricular tachycardia, an arrhythmic condition in which both VF and VT are present). Accordingly, a method of treating a cardiac arrhythmia in a subject in need thereof is provided herein, as described herein. Signs and symptoms of SCD additionally include light-headedness, loss of consciousness, dizziness, shortness of breath. The invention additionally provides a method of treating these signs and symptoms in a subject.

The invention furthermore provides a method of treating a subject at risk for sudden cardiac death. The method comprises the step of administering to the subject a c-Src inhibitor.

Most cases of SCD are related to cardiac arrhythmias, e.g., ventricular arrhythmias. Ventricular fibrillation is the most common cause of SCD and heart attack is the most common cause of ventricular fibrillation. Less common causes of SCD include respiratory arrest (e.g., loss of breathing function), choking, trauma, electrocution, drowning. In exemplary embodiments, the subject at risk for SCD is a subject at risk for SCD caused by ventricular fibrillation or heart attack, a subject suffering from ventricular fibrillation, or a subject suffering from a heart attack. In exemplary embodiments, the subject at risk for SCD is not a subject suffering from respiratory arrest (e.g., loss of breathing function), choking, trauma, electrocution, or drowning.

In exemplary embodiments, the subject at risk for SCD is a subject with a structural abnormality of the heart. The term “structural abnormality of the heart” as used herein is synonymous with “structural heart disease” and refers to any disease or medical condition that affects the heart muscle or changes the architecture of the heart. In some aspects, the structural abnormality of the heart is selected from the group consisting of: ischemic heart disease, chronic stable angina, chronic angina, ischemia, ischemia-reperfusion, coronary artery occlusion-reperfusion, myocardial injury, myocardial toxicity, myocardial infarction, congenital heart lesion, valvular stenosis, valvular regurgitation, coronary artery disease, chronic angina, chronic stable angina, and myocarditis.

In exemplary embodiments, the subject at risk for SCD is a subject with a medical history of or a subject suffering from myocardial infarction (MI). In exemplary aspects, the subject at risk for SCD is a subject which exhibits premature ventricular contractions (PVCs), e.g., multiform PVCs, short coupling intervals, or ventricular tachycardia. In exemplary embodiments, the subject at risk for SCD is a subject with a medical history of or a subject suffering from any one or more of: coronary heart disease, hypertrophic cardiomyopathy, dilated cardiomyopathy, a valvular disease (e.g., aortic stenosis), an acute cardiac illness (e.g., myocarditis), inflammation and/or fibrosis of the myocardium, or left ventricular dysfunction. In exemplary embodiments, the subject at risk for SCD is a subject with a medical history of or a subject suffering from congestive heart failure with an ejection fraction less than 30%. In exemplary embodiments, the subject at risk for SCD is a subject suffering from a combination of regional ischemia, left ventricular dysfunction, and transient inciting events (e.g., worsened ischemia, acidosis, hypoxemia, wall tension, metabolic disturbances, drugs). In exemplary embodiments, the subject at risk for SCD is a subject in the convalescent phase after myocardial infarction or is a subject who survived cardiac arrest.

In exemplary embodiments, the subject at risk for SCD does not suffer from a structural heart abnormality. In exemplary embodiments, the subject has a normal left ventricular ejection fraction (e.g., a left ventricular ejection fraction of about 45% or more), does not suffer from ventricular fibrosis, does not suffer from hypertension, or a combination thereof. In exemplary embodiments, the subject is asystolic. In exemplary aspects, the subject exhibits cardiac oxidative stress, abnormal gap junction function, impaired (e.g., slowed) cardiac conduction (e.g., slow conduction velocity), reduced Connexin 43 levels (e.g., reduced ventricular Connexin 43 levels), increased RAS activation, increased angiotensin II levels, increased angiotensin converting enzyme (ACE) (e.g., increased ACE levels in cardiac cells), increased c-Src levels (e.g., increased total c-Src levels, increased phospho-c-Src levels), reduced myocyte coupling, or a combination of the foregoing.

In exemplary embodiments, the subject at risk for SCD is a male human. In exemplary embodiments, the subject at risk for SCD is a subject aged 45-75 years. In exemplary embodiments, the subject at risk for SCD is a subject who smokes cigarettes. In exemplary embodiments, the subject at risk for SCD is a subject suffering from any one or more of: dyslipidemia, hypertension, diabetes, obesity. In exemplary embodiments, the subject at risk for SCD is a subject suffering from long QT syndrome, Wolff-Parkinson-White syndrome, or Brugada syndrome. In exemplary embodiments, the subject at risk for SCD is a subject suffering from pulmonary embolism, aortic dissetion or aneurismal rupture.

In exemplary embodiments, the subject at risk for SCD suffers from a cardiac arrhythmia at the time of administering the c-Src inhibitor. In exemplary aspects, the cardiac arrhythmia is any one described herein, e.g., a ventricular arrhythmia (e.g., a ventricular tachycardia (VT), ventricular fibrillation (VF), or an arrhythmic condition in which both VT and VF are present). In exemplary embodiments, the subject does not suffer from a cardiac arrhythmia at the time of administering the c-Src inhibitor.

In exemplary embodiments, the subject has a medical history of cardiac arrhythmia, e.g., a ventricular arrhythmia (e.g., a ventricular tachycardia (VT), ventricular fibrillation (VF), or an arrhythmic condition in which both VT and VF are present). In exemplary embodiments, the subject does not have a medical history of a cardiac arrhythmia, e.g., a ventricular arrhythmia (e.g., a ventricular tachycardia (VT), ventricular fibrillation (VF), or an arrhythmic condition in which both VT and VF are present). In exemplary embodiments, the subject is at risk for a cardiac arrhythmia, as described herein. In exemplary aspects, the subject is any of the subject described herein. See, e.g., the section entitled “Cardiac Arrhythmias.”

In exemplary aspects, the subject is a subject exhibiting increased levels of RAS activity (e.g., increased levels of ACE or angiotensin II), as described herein. In exemplary embodiments, the subject is one suffering from one or more of the medical conditions described herein. See, e.g., the section entitled “Ras activation and related medical conditions.”

Cardiac Arrhythmias

As used herein, the term “cardiac arrhythmia” is synonymous with “cardiac dysrhythmia” or “arrhythmia” and refers to any condition in which there is abnormal electrical activity in the heart. The invention provides a method of treating or preventing a cardiac arrhythmia in a subject in need thereof. The method comprises the step of administering to the subject a c-Src inhibitor in an amount effective to treat or prevent the cardiac arrhythmia. In exemplary aspects, the subject is suffering from a cardiac arrhythmia at the time of administration of the c-Src inhibitor. In exemplary aspects, the subject has a medical history of a cardiac arrhythmia or is a subject at risk for a cardiac arrhythmia, as further described herein.

Because the term “prevent” and words stemming therefrom encompasses delaying the onset of the medical condition being prevented, the invention also provides a method of delaying the onset of a cardiac arrhythmia in a subject. The method comprises the step of administering to the subject a c-Src inhibitor in an amount effective to delay the onset of the cardiac arrhythmia in a subject. In exemplary aspects, the method delays the onset of the cardiac arrhythmia by 1 day, 2 days, 4 days, 6 days, 8 days, 10 days, 15 days, 30 days, two months, 4 months, 6 months, 1 year, 2 years, 4 years, or more. In exemplary aspects, delaying the onset of the cardiac arrhythmia is a delay in the first time occurrence of the cardiac arrhythmia or is a delay in a reoccurrence of the cardiac arrhythmia.

Because the term “prevent” and words stemming therefrom encompasses reducing the risk of the medical condition being prevented, the invention additionally provides a method of reducing the risk of a cardiac arrhythmia in a subject is provided herein. The method comprises the step of administering to the subject a c-Src inhibitor in an amount effective to reduce the risk of the cardiac arrhythmia. In exemplary aspects, the method reduces the risk of the cardiac arrhythmia 2-fold, 5-fold, 10-fold, 20-fold, 50-fold, 100-fold, or more.

As used herein, the term “treat” and words stemming therefrom encompasses treatment of a sign or symptom of the medical condition being treated. Accordingly, the invention furthermore provides a method of treating a sign or symptom of a cardiac arrhythmia in a subject in need thereof. The method comprises the step of administering to the subject a c-Src inhibitor in an amount effective to treat the sign or symptom. Signs and symptoms of a cardiac arrhythmia include, for example, palpitations, light-headedness, dizziness, loss of consciousness, shortness of breath.

The invention also provides a method of preventing a medical condition which results from a cardiac arrhythmia, if the cardiac arrhythmia is left untreated. In exemplary aspects, the medical condition is fainting, dizziness, lightheadedness, embolism, stroke, heart failure, or sudden cardiac death. The method comprises the step of administering to the subject a c-Src inhibitor in an amount effective to prevent the medical condition, e.g., SCD.

With regard to the methods of the invention relating to a cardiac arrhythmia, the cardiac arrhythmia in exemplary aspects is a ventricular arrhythmia, such as ventricular fibrillation, ventricular tachycardia, or an arrhythmic condition in which both ventricular fibrillation and ventricular tachycardia are present. In exemplary aspects, the cardiac arrhythmia is an atrial arrhythmia, e.g., an atrial fibrillation, atrial tachycardia, or an arrhythmic condition in which both atrial fibrillation and atrial tachycardia are present. Other types of cardiac arrhythmias are described below.

In exemplary aspects, the cardiac arrhythmia is characterized by an abnormal heart rate. In exemplary aspects, the cardiac arrhythmia is characterized by a bradycardia or a tachycardia.

Bradycardia

In exemplary aspects, the cardiac arrhythmia is a bradycardia in which the resting heart rate is slower than normal. In exemplary aspects, the bradycardia is characterized by a resting heart rate in an adult human which is slower than 60 beats per minute. In exemplary aspects, the bradycardia is a sinus bradycardia. In exemplary aspects, the bradycardia is caused by sinus arrest or AV block or heart block. In exemplary aspects, the bradycardia is cause by a slowed electrical conduction in the heart. In exemplary aspects, the bradycardia is not the bradycardia which is exhibited by the normally functioning heart of an athlete or athletic person.

Tachycardia

In exemplary aspects, the cardiac arrhythmia is a tachycardia in which the resting heart rate is faster than normal. In exemplary aspects, the tachycardia is characterized by a resting heart rate in an adult human which is faster than 100 beats per minute. In exemplary aspects, the tachycardia is a sinus tachycardia. In exemplary aspects, the sinus tachycardia is not caused by physical exercise, emotional stress, hyperthyroidism, ingestion or injection of substances, such as caffeine or amphetamines. In exemplary aspects, the tachycardia is not a sinus tachycardia, e.g., a tachycardia resulting from automaticity, reentry (e.g., fibrillation), or triggered activity. In exemplary aspects, the tachycardia is caused by a slowed electrical conduction in the heart. In exemplary aspects, the tachycardia is caused by an ectopic focus. In exemplary aspects, the tachycardia is combined with abnormal rhythm.

In exemplary aspects, the cardiac arrhythmia is characterized by the mechanism by which it occurs. In exemplary aspects, the cardiac arrhythmia is caused by automaticity, re-entry, or fibrillation.

Automaticity

In exemplary aspects, the cardiac arrhythmia is an abnormal rhythm or a tachycardia caused by automaticity, a condition in which a cardiac muscle cell other than a cardiac muscle of the conduction system fires an impulse of its own. In exemplary aspects, the cardiac arrhythmia is caused by a muscle cell, other than a cell of the sino-atrial (SA) node, atrial-ventricular (AV) node, Bundle of His, or Purkinje fibers, firing an impulse of its own.

Re-Entry

In exemplary aspects, the cardiac arrhythmia is a re-entry arrhythmia in which an electrical impulse recurrently travels in a circle within the heart, rather than moving from one end of the heart to the other and then stopping. In exemplary aspects, the cardiac arrhythmia is a cardiac flutter, a paroxysmal supraventricular tachycardia, or a ventricular tachycardia.

Fibrillation

In exemplary aspects, the cardiac arrhythmia is a fibrillation. In exemplary aspects, the fibrillation is an atrial fibrillation. In exemplary aspects, the fibrillation is a ventricular fibrillation.

Triggered Beats

In exemplary aspects, the cardiac arrhythmia is a triggered beat that occurs when ion channels in the heart cells malfunction, resulting in abnormal propagation of electrical activity and possibly leading to abnormal rhythm.

In exemplary embodiments, the cardiac arrhythmia is classified by site of origin. In exemplary aspects, the cardiac arrhythmia is an atrial arrhythmia (e.g., premature atrial contraction, wandering atrial pacemaker, multifocal atrial tachycardia, atrial flutter, atrial fibrillation). In exemplary aspects, the cardiac arrhythmia is a junction arrhythmia (e.g., supraventricular tachycardia, AV nodal reentral tachycardia, paroxysmal supraventricular tachycardia, junctional rhythm, junctional tachycardia, premature junctional complex). In exemplary aspects, the cardiac arrhythmia is an atrio-ventricular arrhythmia (e.g., AV reentrant tachycardia). In exemplary aspects, the cardiac arrhythmia is a ventricular arrhythmia (e.g., premature ventricular contraction or ventricular extra beat, accelerated idoventricular rhythm, monomorphic ventricular tachycardia, polymorphic ventricular tachycardia, ventricular fibrillation). In exemplary aspects, the cardiac arrhythmia is a heart block (e.g., first degree heart block, Type I second degree heart block, Type 2 second degree heart block, third degree heat block). In exemplary aspects, the cardiac arrhythmia is a premature contraction.

In exemplary aspects, the cardiac arrhythmia is a condition in which two or more types of cardiac arrhythmias are present. In exemplary aspects, the cardiac arrhythmia is a condition in which both ventricular tachycardia and ventricular fibrillation are present. In exemplary aspects, the cardiac arrhythmia is a condition in which a bradycardia is not present.

In exemplary aspects, the cardiac arrhythmia is associated with or caused by cardiac oxidative stress, abnormal gap junction function, impaired (e.g., slowed) cardiac conduction (e.g., slow conduction velocity), reduced Connexin 43 levels (e.g., reduced ventricular Connexin 43 levels), increased RAS activation, increased angiotensin II levels, increased angiotensin converting enzyme (ACE) (e.g., increased ACE levels in cardiac cells), increased c-Src levels (e.g., increased total c-Src levels, increased phospho-c-Src levels), reduced myocyte coupling, or a combination of the foregoing.

With regard to the methods of the invention relating to a cardiac arrhythmia, the subject in exemplary embodiments suffers from a cardiac arrhythmia at the time of administering the c-Src inhibitor. In exemplary aspects, the subject suffers from any of the aforementioned arrythmias, e.g., a ventricular arrhythmia (e.g., a ventricular tachycardia (VT), ventricular fibrillation (VF), or an arrhythmic condition in which both VT and VF are present) at the time of administering the c-Src inhibitor. In exemplary embodiments, the subject does not suffer from a cardiac arrhythmia at the time of administering the c-Src inhibitor. Methods of diagnosing a cardiac arrhythmia in a subject are known in the art and include, for example, conducting an electrocardiogram or an electrophysiology study, which may include measurement of RR intervals.

In exemplary embodiments, the subject has a medical history of cardiac arrhythmia, e.g., a ventricular arrhythmia (e.g., a ventricular tachycardia (VT), ventricular fibrillation (VF), or an arrhythmic condition in which both VT and VF are present). In exemplary embodiments, the subject does not have a medical history of a cardiac arrhythmia, e.g., a ventricular arrhythmia (e.g., a ventricular tachycardia (VT), ventricular fibrillation (VF), or an arrhythmic condition in which both VT and VF are present).

In exemplary embodiments, the subject is at risk for a cardiac arrhythmia. In exemplary aspects, the subject is a subject exhibiting increased levels of RAS activation. In exemplary aspects, the subject is a subject exhibiting increased levels of ACE (e.g., ACE protein, ACE activity, ACE mRNA) or angiotensin II (e.g., angiotensin II protein, angiotensin II mRNA, angiotensin II activity). In exemplary aspects, the subject is any of the subject described in the section entitled “Ras activation and related medical conditions.”

In exemplary aspects, the subject is a subject at risk for SCD. In exemplary aspects, the subject is any of the subjects described in the section entitled “Sudden cardiac death.” For example, in exemplary aspects, the subject is a subject with a structural abnormality of the heart. In other exemplary aspects, the subject is a subject does not suffer from a structure heart abnormality. In exemplary aspects, the subject has a normal left ventricular ejection fraction (e.g., a left ventricular ejection fraction of about 45% or more), does not suffer from ventricular fibrosis, does not suffer from hypertension, or a combination thereof. In exemplary embodiments, the subject is asystolic. In exemplary aspects, the subject exhibits cardiac oxidative stress, abnormal gap junction function, impaired (e.g., slowed) cardiac conduction (e.g., slow conduction velocity), reduced Connexin 43 levels (e.g., reduced ventricular Connexin 43 levels), increased RAS activation, increased angiotensin II levels, increased angiotensin converting enzyme (ACE) (e.g., increased ACE levels in cardiac cells), increased c-Src levels (e.g., increased total c-Src levels, increased phospho-c-Src levels), reduced myocyte coupling, or a combination of the foregoing.

Augmenting Gap Junction Function

The invention provides a method of augmenting or improving gap junction function in a subject in need thereof. The method comprises the step of administering to the subject a c-Src inhibitor in an amount effective to augment gap junction function in the subject.

In exemplary aspects, the subject is a subject exhibiting increased levels of RAS activation. In exemplary aspects, the subject is a subject exhibiting increased levels of ACE (e.g., ACE protein, ACE activity, ACE mRNA) or angiotensin II (e.g., angiotensin II protein, angiotensin II mRNA, angiotensin II activity). In exemplary aspects, the subject is any of the subject described in the section entitled “Ras activation and related medical conditions.”

In exemplary aspects, the subject is a subject at risk for SCD. In exemplary aspects, the subject is any of the subjects described in the section entitled “Sudden cardiac death.” For example, in exemplary aspects, the subject is a subject with a structural abnormality of the heart. In other exemplary aspects, the subject is a subject does not suffer from a structure heart abnormality. In exemplary aspects, the subject has a normal left ventricular ejection fraction (e.g., a left ventricular ejection fraction of about 45% or more), does not suffer from ventricular fibrosis, does not suffer from hypertension, or a combination thereof. In exemplary embodiments, the subject is asystolic. In exemplary aspects, the subject exhibits cardiac oxidative stress, abnormal gap junction function, impaired (e.g., slowed) cardiac conduction (e.g., slow conduction velocity), reduced Connexin 43 levels (e.g., reduced ventricular Connexin 43 levels), increased RAS activation, increased angiotensin II levels, increased angiotensin converting enzyme (ACE) (e.g., increased ACE levels in cardiac cells), increased c-Src levels (e.g., increased total c-Src levels, increased phospho-c-Src levels), reduced myocyte coupling, or a combination of the foregoing.

In exemplary embodiments, the subject suffers from a cardiac arrhythmia, e.g., a ventricular arrhythmia (e.g., a ventricular tachycardia (VT), ventricular fibrillation (VF), or an arrhythmic condition in which both VT and VF are present) at the time of administering the c-Src inhibitor. In exemplary embodiments, the subject does not suffer from a cardiac arrhythmia at the time of administering the c-Src inhibitor. Methods of diagnosing a cardiac arrhythmia in a subject are known in the art and include, for example, conducting an electrocardiogram or an electrophysiology study, which may include measurement of RR intervals.

In exemplary embodiments, the subject has a medical history of cardiac arrhythmia, e.g., a ventricular arrhythmia (e.g., a ventricular tachycardia (VT), ventricular fibrillation (VF), or an arrhythmic condition in which both VT and VF are present). In exemplary embodiments, the subject does not have a medical history of a cardiac arrhythmia, e.g., a ventricular arrhythmia (e.g., a ventricular tachycardia (VT), ventricular fibrillation (VF), or an arrhythmic condition in which both VT and VF are present).

Methods of analyzing gap junction function are known in the art and include but not limited to the assay described herein within Example 1.

Increasing Connexin 43 Levels

In exemplary aspects, the subject exhibits increased levels of RAS activation (e.g., a subject exhibiting increased levels of ACE or angiotensin II).

In exemplary aspects, the subject is a subject exhibiting increased levels of RAS activation. In exemplary aspects, the subject is a subject exhibiting increased levels of ACE (e.g., ACE protein, ACE activity, ACE mRNA) or angiotensin II (e.g., angiotensin II protein, angiotensin II mRNA, angiotensin II activity). In exemplary aspects, the subject is any of the subject described in the section entitled “Ras activation and related medical conditions.”

In exemplary aspects, the subject is a subject at risk for SCD. In exemplary aspects, the subject is any of the subjects described in the section entitled “Sudden cardiac death.” For example, in exemplary aspects, the subject is a subject with a structural abnormality of the heart. In other exemplary aspects, the subject is a subject does not suffer from a structure heart abnormality. In exemplary aspects, the subject has a normal left ventricular ejection fraction (e.g., a left ventricular ejection fraction of about 45% or more), does not suffer from ventricular fibrosis, does not suffer from hypertension, or a combination thereof. In exemplary aspects, the subject exhibits cardiac oxidative stress, abnormal gap junction function, impaired (e.g., slowed) cardiac conduction (e.g., slow conduction velocity), reduced Connexin 43 levels (e.g., reduced ventricular Connexin 43 levels), increased RAS activation, increased angiotensin II levels, increased angiotensin converting enzyme (ACE) (e.g., increased ACE levels in cardiac cells), increased c-Src levels (e.g., increased total c-Src levels, increased phospho-c-Src levels), reduced myocyte coupling, or a combination of the foregoing.

In exemplary embodiments, the subject suffers from a cardiac arrhythmia, e.g., a ventricular arrhythmia (e.g., a ventricular tachycardia (VT), ventricular fibrillation (VF), or an arrhythmic condition in which both VT and VF are present) at the time of administering the c-Src inhibitor. In exemplary embodiments, the subject does not suffer from a cardiac arrhythmia at the time of administering the c-Src inhibitor. Methods of diagnosing a cardiac arrhythmia in a subject are known in the art and include, for example, conducting an electrocardiogram or an electrophysiology study, which may include measurement of RR intervals.

In exemplary embodiments, the subject has a medical history of cardiac arrhythmia, e.g., a ventricular arrhythmia (e.g., a ventricular tachycardia (VT), ventricular fibrillation (VF), or an arrhythmic condition in which both VT and VF are present). In exemplary embodiments, the subject does not have a medical history of a cardiac arrhythmia, e.g., a ventricular arrhythmia (e.g., a ventricular tachycardia (VT), ventricular fibrillation (VF), or an arrhythmic condition in which both VT and VF are present).

In exemplary aspects, the Connexin 43 levels are cardiac Connexin 43 levels, e.g., ventricular Connexin 43 levels, Connexin 43 levels in the gap junction.

Methods of measuring Connexin 43 levels are known in the art and include but not limited to immunoassays (e.g., Western blotting, immunohistochemistry, immunofluorescence), Northern blotting, quantitative PCR, and the like. See, e.g., Sambrook et al., Molecular Cloning: a laboratory manual, 3^{rd} edition, Cold Spring Harbor Laboratory Press, Cold Spring Harbor, N.Y., 2001.

Increasing Cardiac Conduction Velocity

The invention further provides a method of increasing conduction velocity in a subject in need thereof. The method comprises the step of administering to the subject a c-Src inhibitor in an amount effective to increase conduction velocity in the subject. As used herein, the term “conduction velocity” refers to the speed with which an electrical impulse is transmitted through excitable cardiac tissue, as in the movement of an action potential through His-Purkinje fibers of the heart. In exemplary aspects, the subject is any of those described herein. In other exemplary aspects, the subject is a subject does not suffer from a structure heart abnormality. In exemplary aspects, the subject has a normal left ventricular ejection fraction (e.g., a left ventricular ejection fraction of about 45% or more), does not suffer from ventricular fibrosis, does not suffer from hypertension, or a combination thereof. In exemplary aspects, the subject exhibits cardiac oxidative stress, abnormal gap junction function, impaired (e.g., slowed) cardiac conduction (e.g., slow conduction velocity), reduced Connexin 43 levels (e.g., reduced ventricular Connexin 43 levels), increased RAS activation, increased angiotensin II levels, increased angiotensin converting enzyme (ACE) (e.g., increased ACE levels in cardiac cells), increased c-Src levels (e.g., increased total c-Src levels, increased phospho-c-Src levels), reduced myocyte coupling, or a combination of the foregoing. Methods of measuring conduction velocity are known in the art and include those described in Castellanos et al., Br Heart J 37: 242-248 (1975).

Treatment, Prevention

As used herein, the terms “treat,” “prevent,” “reduce” and “increase” as well as words stemming therefrom, do not necessarily imply 100% or complete treatment, prevention, reduction, or increase. Rather, there are varying degrees of treatment, prevention, reduction, or increase of which one of ordinary skill in the art recognizes as having a potential benefit or therapeutic effect. Accordingly, the methods of the invention can provide any amount or any level of treatment of a medical condition, e.g., a cardiac condition, cardiac arrhythmia, (e.g., a ventricular arrhythmia). Furthermore, the treatment or prevention provided by the methods of the invention can include treatment or prevention of one or more conditions or symptoms of the medical condition being treated or prevented. Also, in exemplary aspects, “prevention” encompasses reducing the risk of, delaying the onset of, and/or reducing the occurrence of the medical condition, or a symptom or condition thereof. Further, in exemplary aspects, a method of preventing also refers to a method of treating a medical condition which leads to the medical condition being prevented. For example, a method of preventing sudden cardiac death encompasses a method of treating a ventricular arrhythmia (e.g., ventricular fibrillation) which leads to sudden cardiac death, if left untreated.

Subjects

In exemplary embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In exemplary aspects, the mammals are of the order Primates, Ceboids, or Simoids (monkeys) or of the order Anthropoids (humans and apes). In exemplary aspects, the subject is a human.

c-Src Inhibitors

In exemplary embodiments of any of the methods of the invention, the c-Src inhibitor is a compound which inhibits c-Src from binding to another protein. In exemplary aspects, the c-Src inhibitor is a compound which inhibits a protein binding to c-Src via the SH2 domain of c-Src, the SH3 domain of c-Src, or the substrate binding domain of c-Src.

In exemplary embodiments of any of the methods of the invention, the c-Src inhibitor is a compound which inhibits c-Src tyrosine kinase activity. In exemplary aspects, the c-Src inhibitor exhibits an IC50 which is about 100 μM or less, e.g., about 50 μM or less, about 25 μM or less, about 20 μM or less, about 15 μM or less, about 10 μM or less, about 5 μM or less, about 2 μM or less, about 1 μM or less. In exemplary aspects, the c-Src inhibitor exhibits an IC50 which is about 100 nM or less, e.g., about 50 nM or less, about 25 nM or less, about 20 nM or less, about 15 nM or less, about 10 nM or less, about 5 nM or less, about 2 nM or less, about 1 nM or less.

In exemplary aspects, the c-Src inhibitor is a heterocyclic adenosine triphospate (ATP) analog. In exemplary aspects, the c-Src inhibitor is selected from the group consisting of: (i) a pyrazolo-[2,3-d]pyrimidine, (ii) pyrrolo-[2,3-d]pyrimidine, (iii), pyrido-[2,3-d]pyrimidine, (iv) quinoline carbonitrile, or (v) olomucine. In exemplary aspects, the c-Src inhibitor comprises a structure of Formula I:

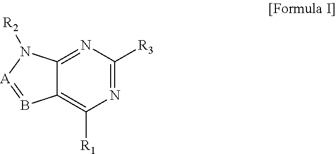

wherein (i) A is a nitrogen atom and B is a carbon atom; (ii) A is a carbon atom and B is a nitrogen atom; or (iii) each of A and B is a carbon atom,

wherein, when B is a carbon atom, B is attached to H, a C1-C4 alkyl, aryl, or a substituted aryl, and R₃ is H;

wherein, when B is a nitrogen atom, R₃ is —NHR₄, wherein R₄ is a C1-C6 alcohol;

wherein R₁ is a —NH₂ or —NHR₅; wherein R₅ is aryl or substituted aryl; and

wherein R₂ is an H, alkyl, aryl, or a substituted aryl.

In exemplary aspects, each of A and B is a carbon atom, B is attached to a substituted aryl, R₁ is a —NH₂, R₂ is a substituted aryl, and R3 is H. In exemplary aspects, B is attached to a substituted aryl of Structure A or B:

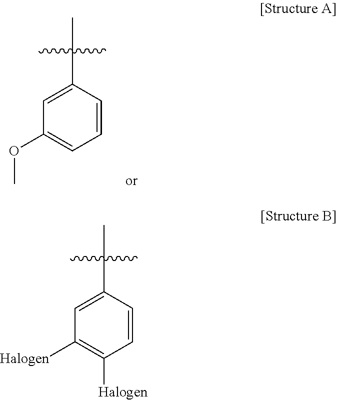

wherein “Halogen” is selected from a group consisting of Cl, F, I, and Br. In exemplary aspects, the halogen is F. In exemplary aspects, R2 is a substituted aryl of Structure C or D:

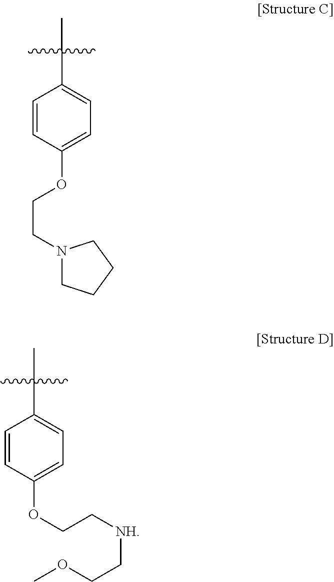

In exemplary aspects, A is a nitrogen atom, B is a carbon atom attached to an aryl, R₁ is —NH₂, R₂ is C(CH₂)₃, and R3 is H.

In exemplary aspects, A is a carbon atom, B is a nitrogen atom, R1 is NHR₅; wherein R₅ is a substituted aryl, R2 is —CH2CH3, and R3 is —NHR₄, wherein R₄ is a C1-C6 alcohol. In exemplary aspects, R₅ is a substituted aryl of Structure D or E:

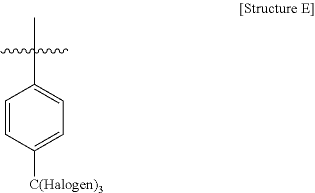

wherein “Halogen” is selected from a group consisting of Cl, F, I, and Br. In exemplary aspects, the halogen is F. In exemplary aspects, R4 is an alcohol of Structure F or G:

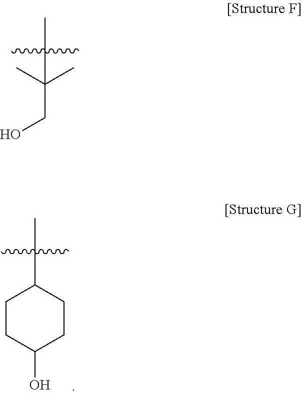

In exemplary aspects, the c-Src inhibitor comprises a structure of Formula II:

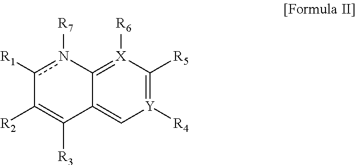

wherein each of X and Y is independently a carbon atom or a nitrogen atom, wherein:

when X is a nitrogen atom, R6 is absent,

when X is a carbon atom, R6 is H, a C1-C4 alkyl, or —O(C1-C4alkyl);

when Y is a nitrogen atom, R4 is absent,

when Y is a carbon atom, R4 is H, C1-C4 alkyl, or —O(C1-C4alkyl);

wherein [symbol] represents a single or double bond, wherein:

when R1 is H, [symbol] represents a double bond,

when R1 is a double bonded oxygen atom, [symbol] represents a single bond; and

wherein:

R2 is —CN or a substituted aryl,

R3 is H or —(CH₂)₀₋₄ aryl, or —(NH)aryl, optionally, wherein “aryl” is a substituted aryl,

R5 is —NHR8 or a substituted alkoxyl,

R7 is absent, H or a C1-C4 alkyl, and

R8 is a substituted aryl.

In exemplary aspects, each of X and Y is a nitrogen atom. In exemplary aspects, R1 is a double bonded oxygen atom, R2 is a substituted aryl, R3 is H, each of R4 and R6 is absent, R5 is NHR8, and R7 is a C1-C4 alkyl. In exemplary aspects, R2 is a substituted aryl of Structure H:

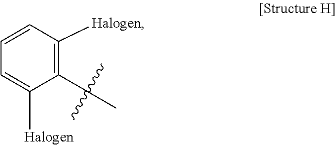

wherein each “Halogen” is as defined herein. In exemplary aspects, each “Halogen” is a chlorine atom. In exemplary aspects, R8 is a substituted aryl of Structure I:

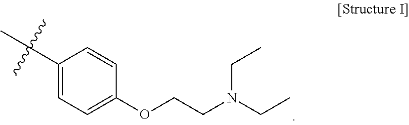

In exemplary aspects, each of X and Y is a carbon atom. In exemplary aspects, R1 is H, R2 is —CN, R3 is —(CH₂)aryl or —(NH)aryl, wherein “aryl” is a substituted aryl, R4 is —O(C1-C4alkyl), R5 is a substituted alkoxyl, R6 is H, and R7 is absent. In exemplary aspects, R3 comprises Structure J:

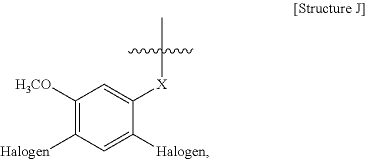

wherein each “Halogen” is as defined herein and X is —(CH2)₀₋₄ or —NH. In exemplary aspects, each “Halogen” is a chlorine atom. In exemplary aspects, R4 is —OCH3. In exemplary aspects, R5 is a substituted alkoxyl comprising the structure of Structure K:

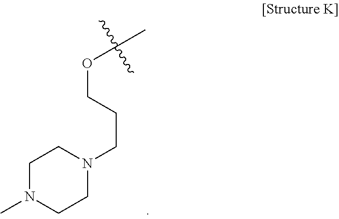

In exemplary embodiments, the c-Src inhibitor comprises a structure of Formula III:

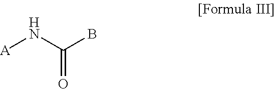

wherein A is an aryl or a substituted aryl, B is a substituted aryl in which at least one of the substitutions comprises (i) a moiety comprising a structure of Structure L:

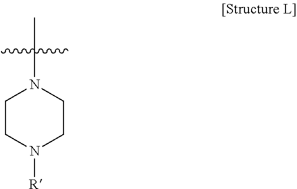

wherein R′ is an alkyl or alkoxy, e.g., a C1-C4 alkyl or C1-C4 alkoxy,

or (ii) a moiety comprising a structure of Structure M:

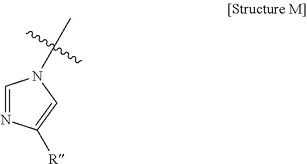

wherein R″ is an alkyl or alkoxy, e.g., a C1-C4 alkyl or C1-C4 alkoxy.

In exemplary embodiments, A of Formula III is a substituted aryl comprising the structure of Structure N:

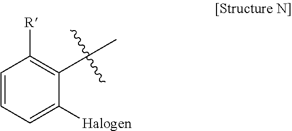

wherein R′ is a C1-C4 alkyl and “halogen” is as described herein. In exemplary embodiments, the halogen is a chlorine atom.

In alternative embodiments, A of Formula III is a substituted aryl comprising Structure O

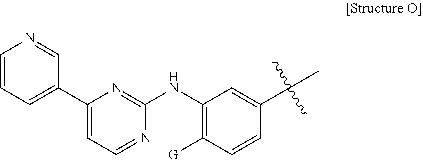

wherein G is —(CH2)₀₋₄—CH₃.

In exemplary aspects, B of Formula III is a substituted aryl comprising the structure of Structure P:

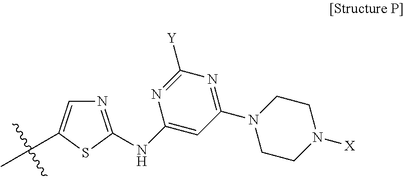

wherein X is —(CH2)₀₋₄OH and Y is —(CH2)₀₋₄CH₃.

In exemplary aspects, B of Formula III is a substituted aryl comprising the structure of Structure Q:

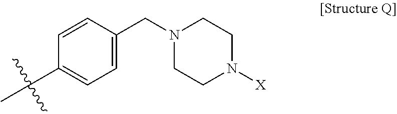

wherein X is —(CH2)₀₋₄CH₃.

In exemplary aspects, B of Formula III is a substituted aryl comprising the structure of Structure R:

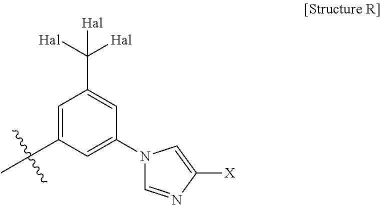

wherein “Hal” is a halogen as described herein and X is X is —(CH2)₀₋₄CH₃. In exemplary aspects, each “Hal” of Structure R is a fluorine atom.

In exemplary embodiments, the c-Src inhibitor comprising a structure of Formula III is selected from the group consisting of imatinib, dasatinib, and nilotinib.

In exemplary aspects, the c-Src inhibitor comprises a structure of Formula IV:

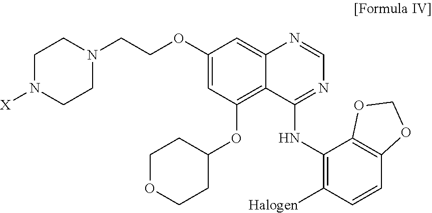

wherein X is (CH₂)₀₋₄CH₃ and “Halogen” is as described herein, e.g., a chlorine atom. In exemplary aspects, the c-Src inhibitor comprising a structure of Formula IV is saracatinib.

In exemplary aspects, the c-Src inhibitor is selected from the group consisting of: PP1, PP2, CGP76030, CGP77675, PD166285, PD173955, PD180970, SKI606, NVP-AAK980, CDP79883, AZD0530, S135, S140, AZM475271, AP23464, AP23451, AP22408, AP23236, dasatinib, bosutinib, and saracatinib.

In exemplary aspects, the c-Src inhibitor is not a compound selected from the group consisting of: PP2, herbimycin A, Src siRNA, 4-amino-5-(4-chlorophenyl)-7(-(t-butyl)pyraxolo[3,4-D]pyrimidine.

Pharmaceutically Acceptable Salts

In exemplary aspects, the c-Src inhibitor is in the form of a salt, e.g., a pharmaceutically acceptable salt. Such salts can be prepared in situ during the final isolation and purification of the active agent or separately prepared by reacting a free base function with a suitable acid. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate.

Basic addition salts also can be prepared in situ during the final isolation and purification of the active agent, or by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like.

Further, basic nitrogen-containing groups can be quaternized with such active agents as lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

Pharmaceutical Compositions

In exemplary embodiments of any of the methods of the invention, the c-Src inhibitor, or pharmaceutically acceptable salt thereof, is formulated into a pharmaceutical composition comprising a pharmaceutically acceptable carrier, diluent, or excipient, and the method comprises administration of the pharmaceutical composition.

Depending on the route of administration, the particular active agent intended for use, as well as other factors, the pharmaceutical composition may comprise additional pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

Accordingly, in exemplary embodiments, the pharmaceutical composition comprises any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, α-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, β-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC) chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil, type II, hydroxyethyl cellulose, 2-hydroxyethyl-β-cyclodextrin, hydroxypropyl cellulose, low-substituted hydroxypropyl cellulose, 2-hydroxypropyl-β-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, potassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-β-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety, discloses various components used in formulating pharmaceutically acceptable compositions and known techniques for the preparation thereof. Except insofar as any conventional agent is incompatible with the pharmaceutical compositions, its use in pharmaceutical compositions is contemplated. Supplementary active ingredients also can be incorporated into the compositions.

Routes of Administration

With regard to any of the methods of the invention, the c-Src inhibitor, pharmaceutically acceptable salt thereof, or the pharmaceutical composition comprising the same, may be administered to the subject by any suitable route of administration. The following discussion on routes of administration is merely provided to illustrate exemplary embodiments and should not be construed as limiting the scope in any way.

Formulations suitable for oral administration can consist of (a) liquid solutions, such as an effective amount of the c-Src inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the c-Src inhibitor in a flavor, usually sucrose and acacia or tragacanth, as well as pastilles comprising the c-Src inhibitor in an inert base, such as gelatin and glycerin, or sucrose and acacia, emulsions, gels, and the like containing, in addition to, such excipients as are known in the art.

The c-Src inhibitors, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the c-Src inhibitor is formulated into a powder blend or into microparticles or nanoparticles. Suitable pulmonary formulations are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The c-Src inhibitor can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the c-Src inhibitor in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the c-Src inhibitor can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the c-Src inhibitor can be formulated as inclusion complexes, such as cyclodextrin inclusion complexes, or liposomes.

Dosages

For purposes herein, the amount or dose of the c-Src inhibitor administered should be sufficient to effect, e.g., a therapeutic or prophylactic response, in the subject or animal over a reasonable time frame. For example, the dose of the c-Src inhibitor should be sufficient to treat a subject exhibiting increased RAS activation, a subject at risk for SCD, or a subject suffering form or at risk for a cardiac arrhythmia, as described herein, in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular c-Src inhibitor and the condition of the animal (e.g., human), as well as the body weight of the animal (e.g., human) to be treated.

Many assays for determining an administered dose are known in the art. For purposes herein, an assay, which comprises comparing the extent to which SCD or ventricular fibrillation is prevented or to which survival is increased upon administration of a given dose of the c-Src inhibitor to a mammal among a set of mammals, each set of which is given a different dose of the c-Src inhibitor, could be used to determine a starting dose to be administered to a mammal. The extent to which SCD or VF is prevented or survival is increased upon administration of a certain dose can be assayed by methods known in the art, including, for instance, the methods described in the EXAMPLES set forth below.

The dose of the c-Src inhibitor also will be determined by the existence, nature and extent of any adverse side effects that might accompany the administration of a particular c-Src inhibitor. Typically, the attending physician will decide the dosage of the c-Src inhibitor with which to treat each individual patient, taking into consideration a variety of factors, such as age, body weight, general health, diet, sex, c-Src inhibitor to be administered, route of administration, and the severity of the condition being treated. By way of example and not intending to limit the invention, the dose of the c-Src inhibitor can be about 0.0001 to about 1 g/kg body weight of the subject being treated/day, from about 0.0001 to about 0.001 g/kg body weight/day, or about 0.01 mg to about 1 g/kg body weight/day.

Controlled Release Formulations

In some embodiments, the c-Src inhibitors described herein can be modified into a depot form, such that the manner in which the c-Src inhibitor is released into the body to which it is administered is controlled with respect to time and location within the body (see, for example, U.S. Pat. No. 4,450,150). Depot forms of c-Src inhibitor can be, for example, an implantable composition comprising the c-Src inhibitor and a porous or non-porous material, such as a polymer, wherein the c-Src inhibitor is encapsulated by or diffused throughout the material and/or degradation of the non-porous material. The depot is then implanted into the desired location within the body of the subject and the c-Src inhibitor is released from the implant at a predetermined rate.

The pharmaceutical composition comprising the c-Src inhibitor in certain aspects is modified to have any type of in vivo release profile. In some aspects, the pharmaceutical composition is an immediate release, controlled release, sustained release, extended release, delayed release, or bi-phasic release formulation. Methods of formulating compounds for controlled release are known in the art. See, for example, Qian et al., J Pharm 374: 46-52 (2009) and International Patent Application Publication Nos. WO 2008/130158, WO2004/033036; WO2000/032218; and WO 1999/040942.

The instant pharmaceutical compositions may further comprise, for example, micelles or liposomes, or some other encapsulated form, or may be administered in an extended release form to provide a prolonged storage and/or delivery effect. The disclosed pharmaceutical formulations may be administered according to any regime including, for example, daily (1 time per day, 2 times per day, 3 times per day, 4 times per day, 5 times per day, 6 times per day), every two days, every three days, every four days, every five days, every six days, weekly, bi-weekly, every three weeks, monthly, or bi-monthly.

Combinations

In some embodiments, the c-Src inhibitor is administered alone, and in alternative embodiments, the c-Src inhibitors are administered in combination with another therapeutic agent which aims to treat or prevent any of the diseases or medical conditions described herein, e.g., SCD, VF. In exemplary embodiments, a c-Src inhibitor of a first structure is co-administered with (simultaneously or sequentially) a c-Src inhibitor of different structure. In alternative or additional embodiments, the c-Src inhibitors described herein may be co-administered with (simultaneously or sequentially) an angiotensin II inhibitor, an anti-arrhythmic agent, an ACE blocker.

In exemplary embodiments, the angiotensin II inhibitor is an angiotensin II receptor antagonist, also known as an angiotensin receptor blocker (ARB), AT₁ receptor antagonist, or a sartan. In exemplary aspects, the ARB is losartan, irbesartan, olmesartan, candesartan, valsartan, telmisartan, EXP 3174, or eprosartan.

In exemplary embodiments, the ACE blocker is a sulfhydryl-containing agent (e.g., captopril, zefenopril), a dicarboxylated-containing agent (e.g., enalapril, ramipril, quinapril, perindopril, lisinopril, benzepril), or a phosphonate-containing agent (e.g., fosinopril). The ACE blocker in some aspects is a casokinin, lactokinin or lactotriopeptide (e.g., Val-Pro-Pro, Ile-Pro-Pro).

In exemplary aspects, the anti-arrhythmic agent is a Class I agent, Class II agent, Class III agent, Class IV agent, or Class V agent, according to the Singh Vaughan Williams classification. In exemplary aspects, the anti-arrhythmic agent is a fast channel blocker, a slow channel blocker, a beta blocker. In exemplary aspects, the anti-arrhythmic agent is quinidine, procainamide, disopyramide, lidocaine, phenyloin, mexiletine, flecamide, propafenone, moricizine, propranolol, esmolol, timolol, metoprolol, atenolol, bisoprolol, amiodarone, sotalol, ibutilide, dofetilide, E-4031, verapimil, diltiazem, adenosine, digoxin.

In exemplary aspects, the c-Src inhibitor is administered with defibrillation, e.g., via an automatic external defibrillator or a precordial thump. In exemplary aspects, the c-Src inhibitor is administered with an implantable defibrillator.

In exemplary aspects, the c-Src inhibitor is co-administered with a therapeutic agent for the treatment of hypertension, including, for example, a thiazide diuretic (e.g., chlorothiazine, hydrochlorothiazide, metolazone), a beta blocker (a.k.a, beta-adrenergic blocking agent (e.g., acebutolol, atenolol, bisoprolol, carvedilol, metoprolol, nadolol, nebivolol, penbutolol, propranolol)), an angiotensin-convertine enzyme (ACE) inhibitor (e.g., benazepril, captopril, enalapril, fosinopril, lisinopril, moexipril, perindopril, quinapril, ramipril, trandolarpil), an angiotensin II receptor blocker (a.k.a., ARBs (e.g., candesartan, eprosartan, irbesartan, losartan, olmesartan, telmisartan, valsartan)), a calcium channel blocker (a.k.a., calcium antagonist, (e.g., amlodipine, diltiazem, felodipine, isradipine, nicardipine, nifedipine, nisoldipine, verapramil)), a rennin inhibitor (e.g., Aliskiren), an alpha blocker (a.k.a, an alpha-adrenergic antagonist, alpha-adrenergic blocking agent, adrenergic blocking agent, alpha-blocking agent, (e.g., doxazosin, prazosin, terazosin, tamsulosin, alfuzosin)), an alpha-beta blocker (a.k.a, alpha-beta adrenergic blocker (e.g., carvedilol, labetalol), a central-acting agent (a.k.a., central adrenergic inhibitor, central alpha agonist, central agonist, (e.g., clonidine, guanfacine, methyldopa)), a vasodilator (e.g., hydralazine, minoxidil).

In alternative or additional embodiments, the c-Src inhibitor is co-administered with (simultaneously or sequentially) a therapeutic agent for the treatment of diabetes or obesity. Anti-diabetic agents known in the art or under investigation include insulin, leptin, Peptide YY (PYY), Pancreatic Peptide (PP), fibroblast growth factor 21 (FGF21), Y2Y4 receptor agonists, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARγ inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; glucokinase activators (GKA); glucagon receptor antagonists (GRA); or FBPase (fructose 1,6-bisphosphatase) inhibitors, GLP-1 agonists.

Anti-obesity agents known in the art or under investigation include appetite suppressants, including phenethylamine type stimulants, phentermine (optionally with fenfluramine or dexfenfluramine), diethylpropion (Tenuate®), phendimetrazine (Prelu-2®, Bontril®), benzphetamine (Didrex®), sibutramine (Meridia®, Reductil®); rimonabant (Acomplia®), other cannabinoid receptor antagonists; oxyntomodulin; fluoxetine hydrochloride (Prozac); Qnexa (topiramate and phentermine), Excalia (bupropion and zonisamide) or Contrave (bupropion and naltrexone); or lipase inhibitors, similar to XENICAL (Orlistat) or Cetilistat (also known as ATL-962), or GT 389-255.

In some embodiments, the active agent is administered in combination with aspirin, or other therapeutic agent which promotes cardiac efficiency.

In view of the foregoing, the invention further provides pharmaceutical compositions and kits additionally comprising one of these other therapeutic agents in combination with the c-Src inhibitor. The additional therapeutic agent may be administered simultaneously or sequentially with the c-Src inhibitor. In some aspects, the c-Src inhibitor is administered before the additional therapeutic agent, while in other aspects, the c-Src inhibitor is administered after the additional therapeutic agent.

The following examples are given merely to illustrate the present invention and not in any way to limit its scope.

### EXAMPLES

### Example 1

This example describes the materials and methods used in the study of the subsequent examples.

Previously, a transgenic mouse model of RAS activation was developed by overexpressing ACE restricted to the heart (ACE8/8 mice) via replacement of the somatic ACE promoter with the cardiac-specific α-myosin heavy chain promoter (9). These mice (ACE8/8) are not hypertensive, have structurally normal hearts with a normal left ventricular ejection fraction (LVEF), and have no ventricular fibrosis. They exhibit cardiac oxidative stress, a high incidence of ventricular tachycardia and ventricular fibrillation (VT/VF), and subsequent sudden cardiac death (SCD) associated with reduced ventricular connexin 43 (Cx43) levels (10).

In the adult heart, ventricular gap junctions are formed primarily by Cx43 protein. A significant reduction in or lack of Cx43 can result in slow conduction velocity and ventricular arrhythmia (11). The molecular mechanism by which RAS activation causes the decrease in Cx43 is unknown, however.

The derivation and electrophysiological characterization of ACE8/8 mice have been described previously (9; 10), and it has been shown by telemetry monitoring that the cause of SCD in those animals are VT/VF, asystole and slowed conduction (10).

Wild-type mice with and without treatment with the c-Src inhibitor 1-(1,1-dimethylethyl)-1-(4-methylphenyl)-1H-pyrazolo[3,4-d]pyrimidin-4-amine (PP1), ACE8/8 mice with and without treatment with PP1, and ACE8/8 mice treated with 1-phenyl-1H-pyrazolo[3,4-d]pyrimidin-4-amine (PP3), an inactive analog of PP1, were studied. The animal experiments were conducted according to the National Institutes of Health (NIH) Guide for the Care and Use of Experimental Animals and were approved by the University of Illinois Institutional Animal Care and Use Committee. Mice of both sexes were treated with 1.5-mg/kg PP1 (Enzo Life Sciences, Plymouth Meeting, Pa.), a specific inhibitor of c-Src tyrosine kinase, twice weekly for 4 consecutive weeks by intraperitoneal injection (21-23). PP3 (Enzo Life Sciences) was administered intraperitoneally at the same dose (1.5 mg/kg twice weekly for 4 consecutive weeks). The treatment of all animals was initiated when the mice were 30 days old.

Electrophysiology Study

The mice were anesthetized with an intraperitoneal injection of ketamine (100 mg/kg) and xylazine (5 mg/kg). After cutdown, a 1.1-F catheter with 0.5-mm interelectrode spacing (EPR 800, Millar Instruments, Houston, Tex.) was placed into the right jugular vein and was advanced into the right ventricle. A constant current stimulator (A320, World Precision Instruments, Sarasota, Fla.) connected to a laptop computer was used for cardiac stimulation. During the experiment, body temperature was maintained at 37° C. with a warming pad. Burst pacing at cycle lengths of 100 to 50 ms was used to test for VT inducibility. A rhythm with more than 3 consecutive ventricular beats was considered to be VT.

Western Blot Analysis

The mice were killed, and their hearts were excised. The ventricular tissue was homogenized in a buffer containing 20 mM of tris-(hydroxymethyl)-aminomethane (Tris-C1) (pH, 7.4), 150 mM of sodium chloride (NaCl), 2.5 mM of ethylenediamine tetraacetic acid (EDTA), 1% Triton-100, 10 μL/mL of phenylmethylsulfonyl fluoride (PMSF), 10 μL/mL of protein inhibitor cocktail (Pierce, Rockford, Ill.), and 10 μL/mL of phosphatase inhibitor cocktail II (Sigma-Aldrich, St. Louis, Mo.). Protein samples (5 to 20 μg) were separated via 10% sodium dodecyl sulfate polyacrylamide gel electrophoresis (SDS-PAGE) and were transferred to nitrocellulose membranes. The membranes were blotted with the primary antibodies against c-Src, phosphorylated (Tyr 416) c-Src, and Cx43 (Cell Signaling, Danvers, Mass.). For a loading control, the membranes were blotted with a primary antibody against glyceraldehyde-3-phosphate dehydrogenase (GAPDH; Santa Cruz Biotech, Santa Cruz, Calif.). After treatment with secondary antirabbit or antimouse antibodies, imaging was performed with enhanced chemiluminescence. The radiographic film images were scanned and analyzed with NIH ImageJ software.

Immunohistochemistry

The mouse hearts were fixed in 10% formalin, after which 8-μm thick sections were blocked for 1 hour at room temperature and were then incubated with anti-Cx43 antibodies overnight at 4° C. at concentrations known to provide the best signal-to-noise ratio. The slides were reviewed with a Zeiss Axioskop microscope (Carl Zeiss, Inc, Thornwood, N.Y.), and photomicrographs with original magnification×40 were taken from the apex, the mid-left ventricle (LV), and the LV base. From each of those sites, photomicrographs were taken from the endocardium and epicardium. The Cx43 content was quantified with the use of a grid that divided the field of view into 200 squares. At the intersection points aligning with the intercalated disks, Cx43 was scored as “1” (present) or “0” (absent). The results were expressed as the percentage occupied by Cx43 in the total area examined, excluding pseudospaces. This method has been used previously to quantify levels of collagen and Cx43 in cardiac tissue (24; 25).

Functional Assessment of Cx43

We used an established technique for measuring Cx43 function that involves fluorescent dye introduction and diffusion in intact heart muscle (26). Fresh hearts were obtained from wild-type, ACE8/8, ACE8/8 PP1-treated, and ACE8/8 PP3-treated mice. A sample from each heart was placed in phosphate buffered saline at 37° C., the anterior surface of the LV was punctured with a 27-gauge needle, and the sample was incubated with a droplet of 0.5% Lucifer yellow (LY) and a droplet of 0.5% Texas Red Dextran (TRD) in 150 mM of LiCl solution. After a 15-minute incubation, the samples were fixed in 4% formaldehyde for 30 minutes, washed in phosphate-buffered saline, frozen in liquid nitrogen, and sliced into 14-μm sections. The sections were mounted on microscope slides and examined on a Leica DM5000 B epifluorescence microscope (Leica Microsystems Inc., Bannockburn, Ill.). Digital images of the spread of LY and TRD were obtained. The measurement of the dye spread was performed with ImageJ software. Molecules of TRD are too large to traverse gap junctions and stains cells with disrupted membrane. The TRD distribution was subtracted from the length of the LY spread at the same site to measure the true LY spread through gap junctions. Dye spread in longitudinal and transverse directions was assessed.

Statistical Analysis

The values are presented as the mean±the SEM. The t test, one-way analysis of variance with post hoc tests of significance, the Tukey honestly significant test, and the Fisher exact test for 2×2 tables were used where appropriate, and a P value of <0.05 was considered statistically significant. The survival data were analyzed with the Kaplan-Meier method, and the value was calculated with the log-rank test. The correlation was assessed with the Pearson correlation coefficient method.

Abbreviations used herein throughout the examples: RAS: Renin-angiotensin system; Ang II: Angiotensin II; ACE: Angiotensin-converting enzyme; LVEF: Left ventricular ejection fraction; LV: Left ventricle; VT: Ventricular tachycardia; VF: Ventricular fibrillation; SCD: Sudden cardiac death; Cx43: Connexin 43; Lucifer yellow: LY; TRD: Texas Red Dextran.

### Example 2

This example demonstrates that c-Src inhibitor treatment prevents SCD and reduces VT inducibility.

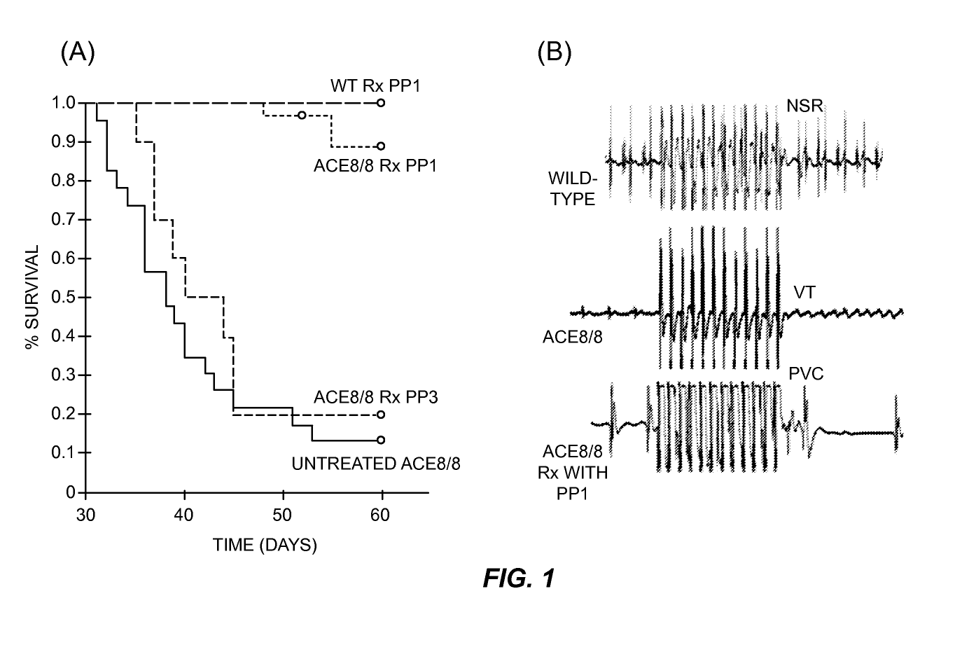

Treatment with PP1 significantly improved the survival rate of ACE8/8 mice from a mean survival time of 10.2±1.5 days to 24.7±0.2 days during the 30 days of treatment and observation (P<0.0001) (FIG. 1). The treatment of wild-type mice with PP1 was not associated with any adverse reaction. The treatment of ACE8/8 mice with PP3, the inactive analog, did not result in a statistically significant improvement in the survival rate when compared with untreated ACE 8/8 mice (11.2±1.2 days vs. 10.2±1.5 days, p=0.09). VT inducibility was observed in 3.3% of the wild-type mice (n=30) and in 86.9% of the ACE8/8 mice (n=23; P=0.0001). PP1-treated ACE8/8 mice showed a significant reduction in VT inducibility (86.9% vs. 50%, P=0.03) (FIG. 1).

### Example 3

This example demonstrates that c-Src inhibitor treatment reduces c-Src and raises Cx43 levels.

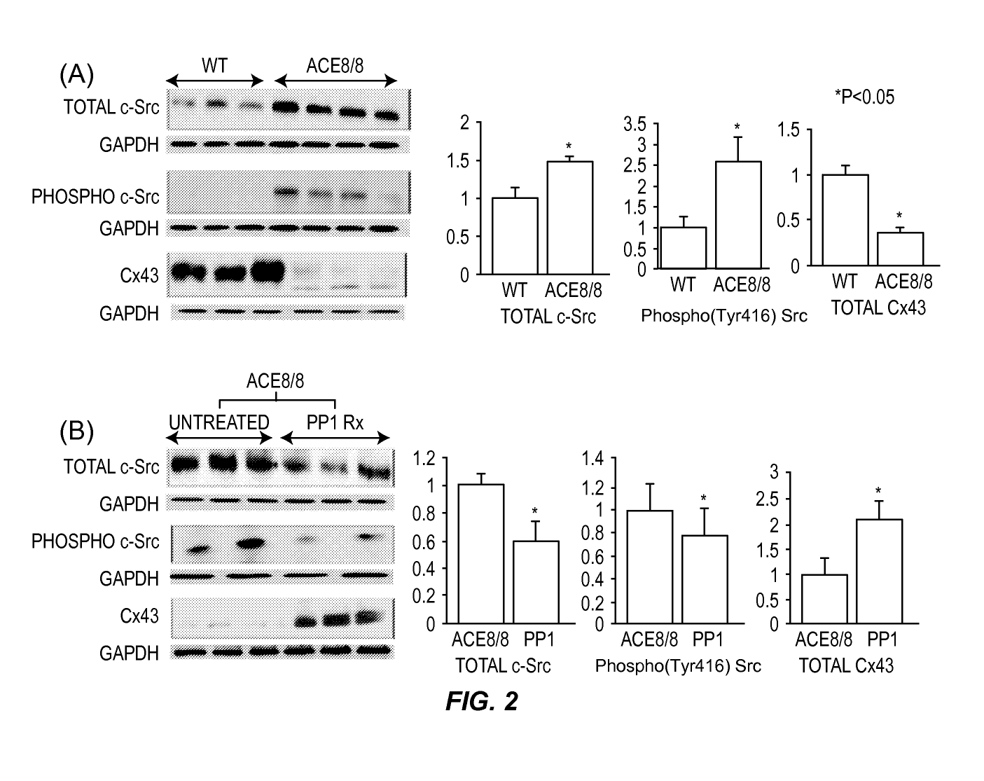

Western blot of the total and phosphorylated (Tyr416) forms of c-Src protein showed a 1.5-fold increase in the total c-Src level and a 2.6-fold increase in the level of phospho-Src protein in the hearts of ACE8/8 mice when compared with those levels in control hearts (P=0.007 and P=0.01, respectively). In untreated ACE8/8 mice, the level of Cx43 protein was 36% of its level in wild-type mice (P=0.0001) (FIG. 2A). In addition, the level of Cx43 was lower in ACE8/8 mice with SCD when compared with those animals that did not experience SCD during the treatment time period (57.8%, P=0.03). PP1 treatment in ACE8/8 mice reduced the total and the phospho-(Tyr416) c-Src protein levels to 58% and 75%, respectively, of those levels in untreated ACE8/8 mice (P=0.03 and P=0.04, respectively) (FIG. 2B). PP1 treatment also caused a 2.1-fold increase in the Cx43 protein level in treated ACE8/8 mice compared with untreated ACE8/8 mice (P=0.002). The correlation between the levels of phospho-(Tyr416) c-Src and Cx43 was statistically significant in ACE8/8 mice (R=−0.85, P=0.01). Treatment of ACE8/8 mice with inactive PP3 did not increase the total Cx43 protein level (P=0.29).

Gene microarray analysis did not show any statistically significant change in the messenger RNA abundances of Cx43.

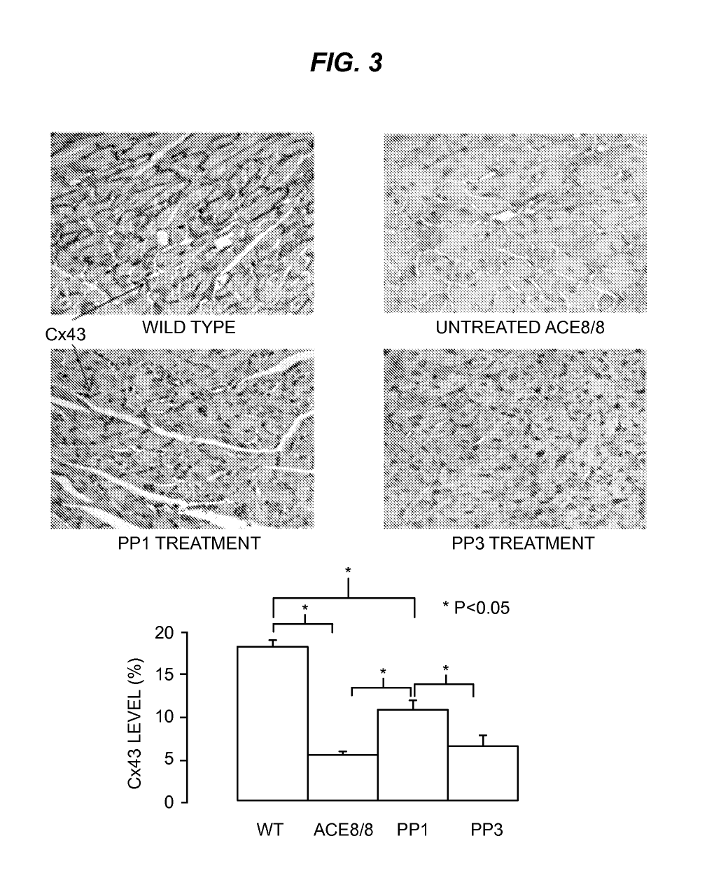

Immunohistochemical analysis showed that the Cx43 level was significantly lower in the ACE8/8 untreated hearts than in the wild-type hearts (5.4±0.7% vs. 18.3±0.6%, P=0.0001) (FIG. 3). PP1 treatment caused a 2.0-fold increase in the Cx43 content in the ACE8/8 mouse hearts (P=0.0005). The extent of that improvement in the Cx43 level after PP1 treatment was consistent with the 2.1-fold increase in the total protein level for Cx43 noted on Western blot analysis. The immunostaining for Cx43 showed that the Cx43 level was increased at intercalated disks. Treatment with the inactive analog PP3 did not increase the Cx43 level at the gap junctions when compared with untreated ACE8/8 mice (P=0.19).

### Example 4

This example demonstrates that c-Src Inhibition improves gap junction function.

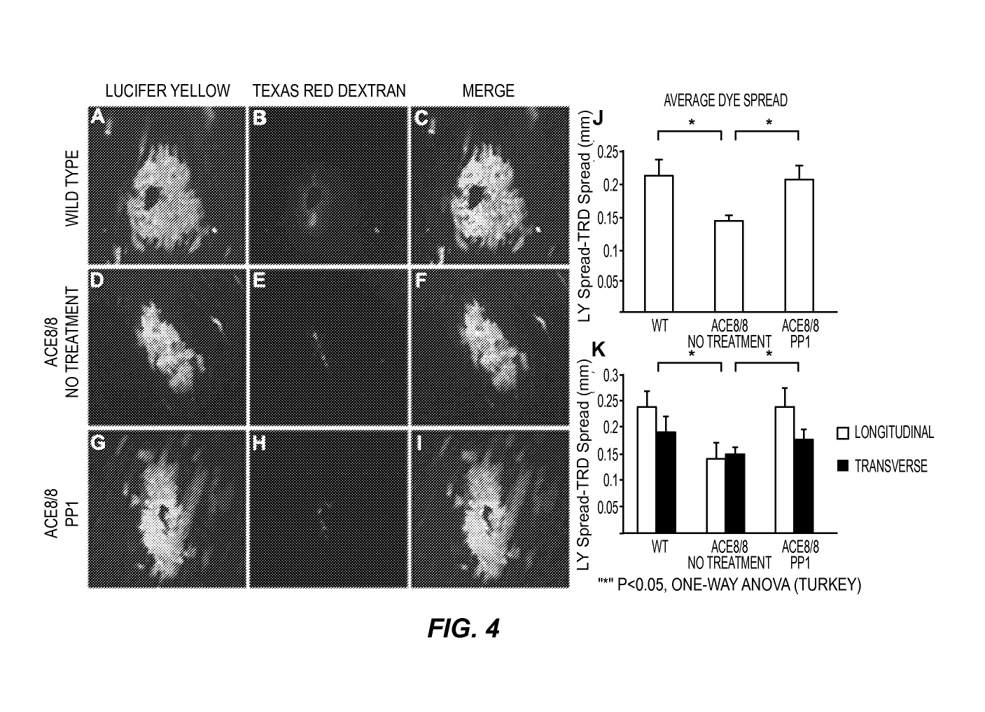

Analysis of dye spread as a functional measure of Cx43 activity showed that LY migration in ACE 8/8 mice was 66% of that in wild-type mice (0.14±0.01 mm vs. 0.21±0.02 mm respectively, P=0.01), which indicates reduced gap junction function in ACE8/8 mice (FIG. 4). Treatment with the c-Src inhibitor PP1 restored gap junction function to that of wild-type mice (0.14±0.01 mm in ACE8/8 vs. 0.21±0.02 mm in PP1-treated ACE8/8 mice, P=0.01; wild-type vs. PP1, P=0.85). The analysis of dye spread in longitudinal and transverse directions separately showed that the changes in dye spread were more prominent in a longitudinal than in a transverse direction. This is consistent with the immunohistochemical result that Cx43 increased most at the intercalated disks. PP3 had no statistically significant effect on dye diffusion.

The foregoing examples demonstrate that elevated RAS activation is associated with c-Src upregulation, Cx43 loss, reduced myocyte coupling, and arrhythmic sudden death, or sudden cardiac death, which can be prevented by c-Src inhibition. These data suggest that an increase in c-Src activity may help mediate RAS-induced arrhythmias and that c-Src inhibitors might exert anti-arrhythmic activity.

We have previously shown that sudden death in this model of cardiac RAS activation results from ventricular arrhythmias and slowed conduction (10). The cardiac sodium channel generates the main current for conduction, but ACES/8 mice have no significant change in their sodium current; however, their level of Cx43 is dramatically reduced (10). Cx43 is the major protein of ventricular gap junctions that are low-resistance conduits for electrical conduction in the heart. In this study, we show that c-Src is upregulated in these animals and that it mediates the effect of Ang II in reducing Cx43 and causing VT/VF.

In the foregoing examples, we show that improvement in the total Cx43 level occurred primarily at the intercalated disks and was correlated with an improvement in gap junction function measured with dye diffusion. The increase in Cx43 and the increase in the ease of dye spread were associated with a reduced risk of VT inducibility and sudden cardiac death. Cx43 reversal was not complete in our study, but the changes noted were in a range consistent with a significant reduction in sudden death.

### Example 5

A population of patients (aged 35-45 years) with a medical history of heart failure is randomized into four groups: one that will receive a c-Src inhibitor, e.g., dasatinib, with an implantable defibrillator, one that will receive the c-Src inhibitor without an implantable defibrillator, one that will receive a placebo and an implantable defibrillator, one that will receive a placebo without an implantable defibrillator. After 6 months time, survival data are collected and analyzed with the Kaplan-Meier method, and the P value was calculated with the log-rank test.

### REFERENCES

The following represents a reference list numbered according to the citation numbering used herein:
[1] Lonn, E. M., Yusuf, S., Jha, P., Montague, T. J., Teo, K. K., Benedict, C. R., and Pitt, B., “Emerging role of angiotensin-converting enzyme inhibitors in cardiac and vascular protection,” Circulation, Vol. 90, No. 4, 1994, pp. 2056-2069.
[2] Pagliaro, P. and Penna, C., “Rethinking the renin-angiotensin system and its role in cardiovascular regulation,” Cardiovasc. Drugs Ther., Vol. 19, No. 1, 2005, pp. 77-87.
[3] Akar, F. G., Spragg, D. D., Tunin, R. S., Kass, D. A., and Tomaselli, G. F., “Mechanisms underlying conduction slowing and arrhythmogenesis in nonischemic dilated cardiomyopathy,” Circ. Res., Vol. 95, No. 7, 2004, pp. 717-725.
[4] Teo, K. K., Mitchell, L. B., Pogue, J., Bosch, J., Dagenais, G., and Yusuf, S., “Effect of ramipril in reducing sudden deaths and nonfatal cardiac arrests in high-risk individuals without heart failure or left ventricular dysfunction,” Circulation, Vol. 110, No. 11, 2004, pp. 1413-1417.
[5] Kober, L., Torp-Pedersen, C., Carlsen, J. E., Bagger, H., Eliasen, P., Lyngborg, K., Videbaek, J., Cole, D. S., Auclert, L., and Pauly, N. C., “A clinical trial of the angiotensin-converting-enzyme inhibitor trandolapril in patients with left ventricular dysfunction after myocardial infarction. Trandolapril Cardiac Evaluation (TRACE) Study Group,” N. Engl. J. Med., Vol. 333, No. 25, 1995, pp. 1670-1676.
[6] Domanski, M. J., Mitchell, G. F., Norman, J. E., Exner, D. V., Pitt, B., and Pfeffer, M. A., “Independent prognostic information provided by sphygmomanometrically determined pulse pressure and mean arterial pressure in patients with left ventricular dysfunction,” J. Am. Coll. Cardiol., Vol. 33, No. 4, 1999, pp. 951-958.
[7] Yusuf, S., Sleight, P., Pogue, J., Bosch, J., Davies, R., and Dagenais, G., “Effects of an angiotensin-converting-enzyme inhibitor, ramipril, on cardiovascular events in high-risk patients. The Heart Outcomes Prevention Evaluation Study Investigators,” N. Engl. J. Med., Vol. 342, No. 3, 2000, pp. 145-153.
[8] Hattori, Y., Atsushi, S., Hiroaki, F., and Toyama, J., “Effects of cilazapril on ventricular arrhythmia in patients with congestive heart failure,” Clin. Ther., Vol. 19, No. 3, 1997, pp. 481-486.
[9] Xiao, H. D., Fuchs, S., Campbell, D. J., Lewis, W., Dudley, S. C., Jr., Kasi, V. S., Hoit, B. D., Keshelava, G., Zhao, H., Capecchi, M. R., and Bernstein, K. E., “Mice with cardiac-restricted angiotensin-converting enzyme (ACE) have atrial enlargement, cardiac arrhythmia, and sudden death,” Am. J. Pathol., Vol. 165, No. 3, 2004, pp. 1019-1032.
[10] Kasi, V. S., Xiao, H. D., Shang, L. L., Iravanian, S., Langberg, J., Witham, E. A., Jiao, Z., Gallego, C. J., Bernstein, K. E., and Dudley, S. C., Jr., “Cardiac-restricted angiotensin-converting enzyme overexpression causes conduction defects and connexin dysregulation,” Am. J. Physiol Heart Circ. Physiol, Vol. 293, No. 1, 2007, pp. H182-H192.
[11] Gutstein, D. E., Morley, G. E., Tamaddon, H., Vaidya, D., Schneider, M. D., Chen, J., Chien, K. R., Stuhlmann, H., and Fishman, G. I., “Conduction slowing and sudden arrhythmic death in mice with cardiac-restricted inactivation of connexin43,” Circ. Res., Vol. 88, No. 3, 2001, pp. 333-339.
[12] Das, J., Chen, P., Norris, D., Padmanabha, R., Lin, J., Moquin, R. V., Shen, Z., Cook, L. S., Doweyko, A. M., Pitt, S., Pang, S., Shen, D. R., Fang, Q., de Fex, H. F., McIntyre, K. W., Shuster, D. J., Gillooly, K. M., Behnia, K., Schieven, G. L., Wityak, J., and Barrish, J. C., “2-aminothiazole as a novel kinase inhibitor template. Structure-activity relationship studies toward the discovery of N-(2-chloro-6-methylphenyl)-2-[[6-[4-(2-hydroxyethyl)-1-piperazinyl)]-2-methyl-4-pyrimidinyl]amino)]-1,3-thiazole-5-carboxamide (dasatinib, BMS-354825) as a potent pan-Src kinase inhibitor,” J. Med. Chem., Vol. 49, No. 23, 2006, pp. 6819-6832.
[13] Hennequin, L. F., Allen, J., Breed, J., Curwen, J., Fennell, M., Green, T. P., Lambert-van der Brempt, C., Morgentin, R., Norman, R. A., Olivier, A., Otterbein, L., Ple, P. A., Warin, N., and Costello, G., “N-(5-chloro-1,3-benzodioxol-4-yl)-7-[2-(4-methylpiperazin-1-yl)ethoxy]-5-(tetrahydro-2H-pyran-4-yloxy)quinazolin-4-amine, a novel, highly selective, orally available, dual-specific c-Src/Abl kinase inhibitor,” J. Med. Chem., Vol. 49, No. 22, 2006, pp. 6465-6488.
[14] Pham, N. A., Magalhaes, J. M., Do, T., Schwock, J., Dhani, N., Cao, P. J., Hill, R. P., and Hedley, D. W., “Activation of Src and Src-associated signaling pathways in relation to hypoxia in human cancer xenograft models,” Int. J. Cancer, Vol. 124, No. 2, 2009, pp. 280-286.
[15] Trevino, J. G., Summy, J. M., Lesslie, D. P., Parikh, N. U., Hong, D. S., Lee, F. Y., Donato, N. J., Abbruzzese, J. L., Baker, C. H., and Gallick, G. E., “Inhibition of SRC expression and activity inhibits tumor progression and metastasis of human pancreatic adenocarcinoma cells in an orthotopic nude mouse model,” Am. J. Pathol., Vol. 168, No. 3, 2006, pp. 962-972.
[16] Wojcik, E. J., Sharifpoor, S., Miller, N. A., Wright, T. G., Watering, R., Tremblay, E. A., Swan, K., Mueller, C. R., and Elliott, B. E., “A novel activating function of c-Src and Stat3 on HGF transcription in mammary carcinoma cells,” Oncogene, Vol. 25, No. 19, 2006, pp. 2773-2784.
[17] Kieken, F., Mutsaers, N., Dolmatova, E., Virgil, K., Wit, A. L., Kellezi, A., Hirst-Jensen, B. J., Duffy, H. S., and Sorgen, P. L., “Structural and molecular mechanisms of gap junction remodeling in epicardial border zone myocytes following myocardial infarction,” Circ. Res., Vol. 104, No. 9, 2009, pp. 1103-1112.
[18] Aikawa, R., Komuro, I., Yamazaki, T., Zou, Y., Kudoh, S., Tanaka, M., Shiojima, I., Hiroi, Y., and Yazaki, Y., “Oxidative stress activates extracellular signal-regulated kinases through Src and Ras in cultured cardiac myocytes of neonatal rats,” J. Clin. Invest, Vol. 100, No. 7, 1997, pp. 1813-1821.
[19] Haendeler, J., Hoffmann, J., Brandes, R. P., Zeiher, A. M., and Dimmeler, S., “Hydrogen peroxide triggers nuclear export of telomerase reverse transcriptase via Src kinase family-dependent phosphorylation of tyrosine 707,” Mol. Cell Biol., Vol. 23, No. 13, 2003, pp. 4598-4610.
[20] Khadaroo, R. G., He, R., Parodo, J., Powers, K. A., Marshall, J. C., Kapus, A., and Rotstein, O. D., “The role of the Src family of tyrosine kinases after oxidant-induced lung injury in vivo,” Surgery, Vol. 136, No. 2, 2004, pp. 483-488.
[21] Liu, Y., Bishop, A., Witucki, L., Kraybill, B., Shimizu, E., Tsien, J., Ubersax, J., Blethrow, J., Morgan, D. O., and Shokat, K. M., “Structural basis for selective inhibition of Src family kinases by PP1,” Chem. Biol., Vol. 6, No. 9, 1999, pp. 671-678.
[22] Amoui, M., Draber, P., and Draberova, L., “Src family-selective tyrosine kinase inhibitor, PP1, inhibits both Fc epsilonRI- and Thy-1-mediated activation of rat basophilic leukemia cells,” Eur. J. Immunol., Vol. 27, No. 8, 1997, pp. 1881-1886.
[23] Hanke, J. H., Gardner, J. P., Dow, R. L., Changelian, P. S., Brissette, W. H., Weringer, E. J., Pollok, B. A., and Connelly, P. A., “Discovery of a novel, potent, and Src family-selective tyrosine kinase inhibitor. Study of Lck- and FynT-dependent T cell activation,” J. Biol. Chem., Vol. 271, No. 2, 1996, pp. 695-701.
[24] Morita, N., Sovari, A. A., Xie, Y., Fishbein, M. C., Mandel, W. J., Garfinkel, A., Lin, S. F., Chen, P. S., Xie, L. H., Chen, F., Qu, Z., Weiss, J. N., and Karagueuzian, H. S., “Increased susceptibility of aged hearts to ventricular fibrillation during oxidative stress,” Am. J. Physiol Heart Circ. Physiol, Vol. 297, No. 5, 2009, pp. H1594-H1605.
[25] Sovari A A, Vandani N, Morita N, Roos K, Weiss J N, and Karagueuzian H S. Oxidative Stress Promotes Ventricular Fibrillation at the Early Stages of Hypertension: Role of Ca²⁺/CaM Kinase-II. Heart Rhythm 7 (suppl.), PO3-92. 5-30-2010.
[26] el-Fouly, M. H., Trosko, J. E., and Chang, C. C., “Scrape-loading and dye transfer. A rapid and simple technique to study gap junctional intercellular communication,” Exp. Cell Res., Vol. 168, No. 2, 1987, pp. 422-430.
[27] Reaume, A. G., de Sousa, P. A., Kulkarni, S., Langille, B. L., Zhu, D., Davies, T. C., Juneja, S. C., Kidder, G. M., and Rossant, J., “Cardiac malformation in neonatal mice lacking connexin43,” Science, Vol. 267, No. 5205, 1995, pp. 1831-1834.
[28] Thomas, S. P., Kucera, J. P., Bircher-Lehmann, L., Rudy, Y., Saffitz, J. E., and Kleber, A. G., “Impulse propagation in synthetic strands of neonatal cardiac myocytes with genetically reduced levels of connexin43,” Circ. Res., Vol. 92, No. 11, 2003, pp. 1209-1216.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed:

1. A method of treating a medical condition in which activation of the renin-angiotensin system (RAS) is increased in a subject in need thereof, the method comprising the step of administering to the subject a c-Src inhibitor in an amount effective to treat the medical condition.

2. The method of claim 1, wherein the medical condition is one in which angiotensin converting enzyme (ACE) or angiotensin II levels is increased.

3. The method of claim 1 or 2, wherein the medical condition is a cardiac condition.

4. The method of claim 3, wherein the cardiac condition is selected from the group consisting of: heart failure, cardiac arrhythmia, low ejection fraction, myocardial infarction, atherosclerosis, and cardiomyopathy.

5. The method of claim 4, wherein the heart failure is systolic heart failure.

6. The method of claim 4, wherein the cardiac arrhythmia is a ventricular arrhythmia.

7. The method of claim 6, wherein the ventricular arrhythmia is a ventricular fibrillation (VF), ventricular tachycardia (VT), or an arrhythmic condition in which both VF and VT are present.

8. The method of claim 4, wherein the low ejection fraction is an ejection fraction of about 45% or less.

9. The method of claim 1 or 2, wherein the medical condition is a metabolic disease or a renal disease.

10. The method of claim 9, wherein the metabolic disease is diabetes, hypertension, or obesity.

11. A method of treating or preventing a cardiac arrhythmia in a subject in need thereof, the method comprising the step of administering to the subject a c-Src inhibitor in an amount effective to treat or prevent the cardiac arrhythmia.

12. The method of claim 11, wherein the cardiac arrhythmia is a ventricular arrhythmia.

13. The method of claim 12, wherein the ventricular arrhythmia is a ventricular fibrillation (VF), a ventricular tachycardia (VT), or an arrhythmic condition in which both VF and VT are present.

14. A method of delaying the onset of sudden cardiac death (SCD) in a subject in need thereof, the method comprising the step of administering to the subject a c-Src inhibitor in an amount effective to delay the onset of SCD.

15. The method of any of claims 1 to 14, wherein the subject (i) has a normal left ventricular ejection fraction, (ii) does not suffer from ventricular fibrosis, (iii) does not suffer from hypertension (iv) is asystolic, (v) exhibits one or more of: cardiac oxidative stress, abnormal gap junction function, impaired cardiac conduction, reduced Connexin 43 levels, increased RAS activation, increased angiotensin II levels, increased ACE levels, increased c-Src levels, reduced myocyte coupling, (vi) or a combination of (i) to (v).

16. The method of any one of the preceding claims, wherein the c-Src inhibitor inhibits c-Src from binding to another protein.

17. The method of any one of the preceding claims, wherein the c-Src inhibitor inhibits tyrosine kinase activity.

18. The method of claim 17, wherein the c-Src inhibitor is a heterocyclic adenosine triphospate (ATP) analog.

19. The method of any one of the preceding claims, wherein the c-Src inhibitor is a (i) pyrazolo-[2,3-d]pyrimidine, (ii) pyrrolo-[2,3-d]pyrimidine, (iii), pyrido-[2,3-d]pyrimidine, (iv) quinoline carbonitrile, or (v) olomucine.

20. The method of any one of the preceding claims, wherein the c-Src inhibitor comprises a structure of Formula I: 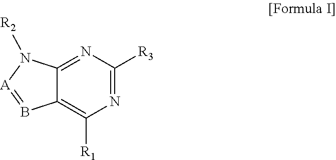
wherein (i) A is a nitrogen atom and B is a carbon atom; (ii) A is a carbon atom and B is a nitrogen atom; or (iii) each of A and B is a carbon atom,
wherein, when B is a carbon atom, B is attached to H, a C1-C4 alkyl, aryl, or a substituted aryl, and R₃ is H;
wherein, when B is a nitrogen atom, R₃ is —NHR₄, wherein R₄ is a C1-C6 alcohol;
wherein R₁ is a —NH₂ or —NHR₅; wherein R₅ is aryl or substituted aryl; and
wherein R₂ is an H, alkyl, aryl, or a substituted aryl.

21. The method of any one of the preceding claims, wherein the c-Src inhibitor comprises a structure of Formula II: 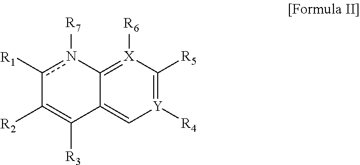
wherein each of X and Y is independently a carbon atom or a nitrogen atom, wherein:
when X is a nitrogen atom, R6 is absent,
when X is a carbon atom, R6 is H, a C1-C4 alkyl, or —O(C1-C4alkyl);
when Y is a nitrogen atom, R4 is absent,
when Y is a carbon atom, R4 is H, C1-C4 alkyl, or —O(C1-C4alkyl);
wherein [symbol] represents a single or double bond, wherein:
when R1 is H, [symbol] represents a double bond,
when R1 is a double bonded oxygen atom, [symbol] represents a single bond; and
wherein:
R2 is —CN or a substituted aryl,
R3 is H or —(CH2)₀₋₄ aryl, or —(NH)aryl, optionally, wherein “aryl” is a substituted aryl,
R5 is —NHR8 or a substituted alkoxyl,
R7 is absent, H or a C1-C4 alkyl, and
R8 is a substituted aryl.

22. The method of any one of the preceding claims, wherein the c-Src inhibitor comprises a structure of Formula III: 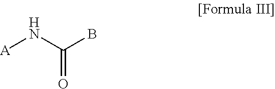
wherein A is an aryl or a substituted aryl, B is a substituted aryl in which at least one of the substitutions comprises (i) a moiety comprising a structure of Structure L: 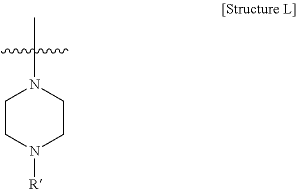
wherein R′ is an alkyl or alkoxy, e.g., a C1-C4 alkyl or C1-C4 alkoxy,
or (ii) a moiety comprising a structure of Structure M: 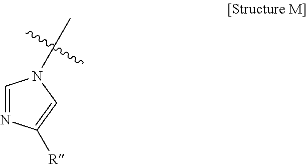
wherein R″ is an alkyl or alkoxy, e.g., a C1-C4 alkyl or C1-C4 alkoxy.

23. The method of any one of the preceding claims, wherein the c-Src inhibitor comprises a structure of Formula IV: 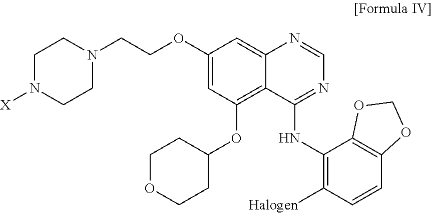
wherein X is (CH₂)₀₋₄CH₃ and “Halogen” is Br, Cl, or F.

24. The method of any one of the preceding claims, wherein the c-Src inhibitor is selected from the group consisting of: PP1, PP2, CGP76030, CGP77675, PD166585, PD173955, PD180970, SKI606, NVP-AAK980, CDP79883, AZD0530, S135, S140, AZM475271, AP23464, AP23451, AP22408, AP23236, dasatinib, imatinib, nilotinib, bosutinib, and saracatinib.
